(19) United States
(12) Patent Application Publication
(10) Pub. No.: US 2025/0115565 A1
(43) Pub. Date: Apr. 10, 2025

(54) SOLID CRYSTALLINE FORMS OF HELICASE-PRIMASE INHIBITORS AND PROCESS OF PREPARATION THEREOF

(71) Applicant: INNOVATIVE MOLECULES GMBH, München (DE)

(72) Inventors: Gerald KLEYMANN, Bad Salzuflen (DE); Christian GEGE, Ehingen (DE)

(73) Assignee: INNOVATIVE MOLECULES GMBH, München (DE)

(21) Appl. No.: 18/729,411

(22) PCT Filed: Jan. 16, 2023
(86) PCT No.: PCT/EP2023/050883

§ 371 Date: Jul. 16, 2024

(30) Foreign Application Priority Data
Jan. 17, 2022 (EP) 22151820.2
Apr. 27, 2022 (EP) 22170332.5

(51) Int. Cl. C07D 277/54; A61K 31/426; C07B 59/00

(52) U.S. Cl. CPC C07D 277/54; A61K 31/426; C07B 59/002

(57) ABSTRACT

The present invention provides solid crystalline forms of compounds useful as helicase-primase inhibitors, compositions thereof, methods of producing the same, and methods of using the same in the treatment of herpes simplex infection and -mediated diseases.

### SUMMARY OF THE INVENTION

The present invention provides solid crystalline forms of antiviral compounds useful as helicase-primase inhibitors, compositions thereof, methods of producing the same, and methods of using the same in the treatment of herpes simplex infections and -mediated diseases.

### BACKGROUND

The pandemic of viral infections has plagued humanity since ancient times, causing mucocutaneous infection such as herpes labialis and herpes genitalis. Disease symptoms often interfere with everyday activities and occasionally HSV infections are the cause of life-threatening (encephalitis) or sight-impairing disease (keratitis), especially in neonates, elderly and the immunocompromised patient population such as transplant or cancer patients or patients with an inherited immunodeficiency syndrome or disease. After infection the alpha herpesviridae persist for life in neurons of the host in a latent form, periodically reactivating and often resulting in significant psychosocial distress for the patient. Currently no cure is available.

So far, vaccines, interleukins, interferones, therapeutic proteins, antibodies, immunomodulators and small-molecule drugs with specific or non-specific modes of action lacked either efficacy or the required safety profile to replace the nucleosidic drugs acyclovir, valacyclovir and famciclovir as the first choice of treatment.

The known aminothiazoles (e.g. pritelivir, HN0037) are the most potent drugs in development today. These antiviral agents act by inhibiting the herpesviral helicase primase, display low resistance rates in vitro and superior efficacy in animal models compared to nucleosidic drugs, however, development is hampered by off-target carbonic anhydrase activity, reduced neuronal tissue and brain penetration and an unusual pharmacokinetic profile.

Herpes viruses are neurotrophic viruses, which means that after infection they enter and settle in neuronal tissue leading to a persisting presence of herpes viruses for life in neurons of the host in a latent form and a permanent neuronal exposure. Such permanent neuronal exposure with latent forms of herpes viruses is the reason for lifelong risk of recurrent and periodically reactivating herpes infections often resulting in significant psychosocial distress for the patient. Such neuronal herpes virus exposure is further the cause of herpesvirus encephalitis (or herpes simplex encephalitis; HSE), which is thought to be caused by the transmission of herpes virus from a peripheral site on the face following HSV-1 reactivation or from neuronal tissue, along a nerve axon to the brain. The virus lies dormant in the ganglion of the trigeminal cranial nerve or in the neuronal tissue and gains access to the brain where it causes HSE. It is therefore important to provide highly active antiviral drugs allowing to treat and eliminate also (dormant) herpes viruses in neuronal tissue and nerves and therewith avoid recurrence and reactivation of herpes infections or even the severe implications like HSE. Known antiviral drugs as e.g. the known aminothiazoles, having insufficient efficacy to enter neuronal tissue or to cross the blood brain barrier to enter the brain are not able to provide an effective and eradicative cure for treating also latent or dormant forms of herpes viruses or even HSE.

This patent application discloses new solid crystalline forms of antiviral aminothiazole compounds with a more suitable pharmacokinetic and stability profile (e.g. due to improved solubility and bioavailability allowing a higher passage of the antiviral drug compound into neuronal tissue and into the brain). Furthermore, the new solid crystalline forms of antiviral aminothiazole compounds are characterized by improved compound stability and improved bioavailability, making them more suitable for pharmaceutical development and use as a medicament.

### PRIOR ART

From the prior art aminothiazoles of the general formula (A)

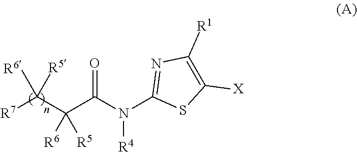

for the use as antiviral compounds are known.

In particular, WO2003/007946 and WO2001/047904 disclose such aminothiazoles (A), wherein X is a sulfonamide moiety. Both documents describe a compound with the following structure

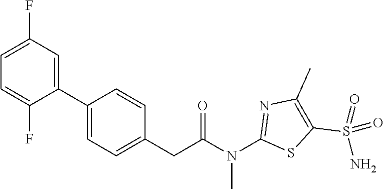 [SMILES: Cc1nc(N(C)C(=O)Cc2ccc(-c3cc(F)ccc3F)cc2)sc1S(N)(=O)=O]

which can be prepared with a method as described in Example 8 of WO2003/007946 and in Example 87 of WO2001/047904 in the form of a yellow solid with a melting point of 184° C.

WO2017/174640 describes thiazolylamides of Formula (A) wherein X is a sulfanimine, sulfinimidamide, sulfoximine or sulfoximidamide.

WO2019/068817 describes enantiomers of the compounds according to WO2017/174640.

WO2020/109389 describes the new use of the aminothiazole compounds according to WO2017/174640 and WO2019/068817 in a combination therapy with oncolytic viruses for treating cancer.

The international application WO2022/090409 describes deuterated analogs of the compounds according to WO2017/174640 and WO2019/068817.

The non-patent literature publication of Gege et al., “A helicase-primase drug candidate with sufficient target tissue exposure affects latent neural herpes simplex virus infections”; Sci. Transl. Med. 2021; 13: eabf8668 describes experimental test results with a variety of antiviral helicase primase inhibitor compounds described in the aforementioned prior art.

In all these patent applications, no solid crystalline forms according to Formula (A) as defined herein

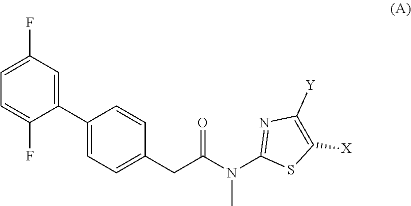

are described or mentioned. In particular, said documents do not specify any particular salt or solid form.

Several properties can be altered by crystallisation or salt formation, such as solubility, dissolution rate, bioavailability, hygroscopicity, flavor, developability and physical/chemical stability.

Given the availability of a large number of pharmaceutically acceptable counterions and the lack of correlation between the nature of a pharmaceutically acceptable counterion with the final properties of the corresponding salt, salt selection process is difficult and its results are, a priori, unpredictable.

There is a need to provide crystalline (salt) forms of antiviral compounds according Formula (A) with improved physicochemical and pharmaceutical properties, without affecting negatively other important parameters, such as hygroscopicity or bioavailability of the active compound with the final goal to obtain an improvement in the production, handling, storage and pharmaceutical properties of said compounds according Formula (A).

### SUMMARY

The present invention relates to novel solid forms of antiviral helicase primase inhibitor compounds of Formula (I):

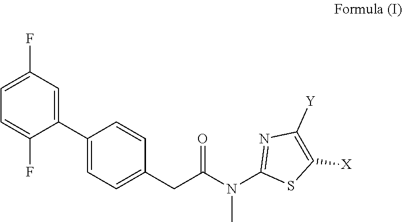

wherein X is selected from

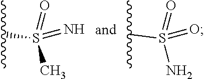

Y is selected from CH₃ and CD₃;
or a pharmaceutically acceptable salt, co-crystal, hydrate or solvate thereof.

These novel forms are useful, for example, for treating human patients suffering from a herpes simplex-mediated disorder. The novel solid forms of the present disclosure can be useful for preparing a medicament for treating herpes simplex virus infections and disease. The novel solid forms of the present disclosure can be used as helicase-primase inhibitors.

In some embodiments, the present disclosure is directed to novel solid forms of the free base compounds with the chemical structure

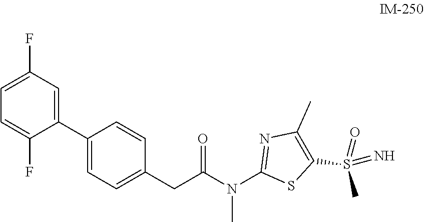 [SMILES: Cc1nc(N(C)C(=O)Cc2ccc(-c3cc(F)ccc3F)cc2)sc1[S@@](C)(=N)=O]

In some embodiments, the present disclosure is directed to a novel solid form of the HCl salt with the chemical structure

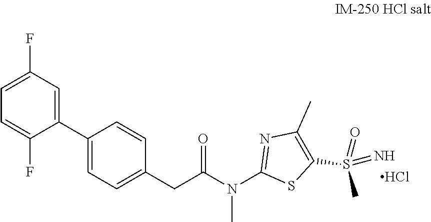 [SMILES: Cc1nc(N(C)C(=O)Cc2ccc(-c3cc(F)ccc3F)cc2)sc1[S@@](C)(=N)=O ; Cl]

In some embodiments, the present disclosure is directed to a novel deuterated solid form of the free base compounds with the chemical structure

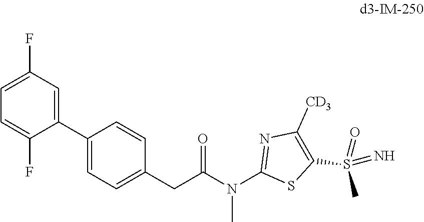 [SMILES: [2H]C([2H])([2H])c1nc(N(C)C(=O)Cc2ccc(-c3cc(F)ccc3F)cc2)sc1[S@@](C)(=N)=O]

In some embodiments, the present disclosure is directed to a novel solid form of the HCl salt with the chemical structure

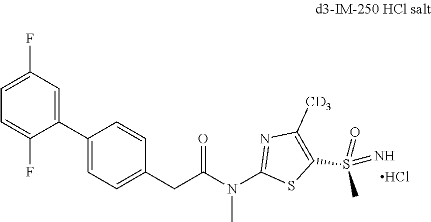 [SMILES: [2H]C([2H])([2H])c1nc(N(C)C(=O)Cc2ccc(-c3cc(F)ccc3F)cc2)sc1[S@@](C)(=N)=O ; Cl]

In some embodiments, the present disclosure is directed to a novel solid form of the napadisylate salt with the chemical structure

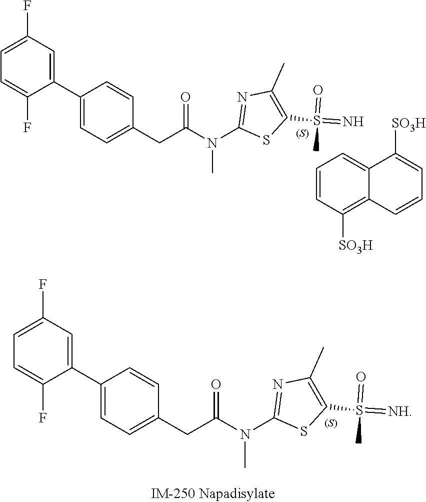

In some embodiments, the present disclosure is directed to a novel solid form with the chemical structure

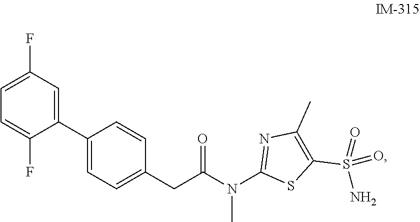 [SMILES: Cc1nc(N(C)C(=O)Cc2ccc(-c3cc(F)ccc3F)cc2)sc1S(N)(=O)=O]

In some embodiments, the present disclosure is directed to a novel solid form with the chemical structure

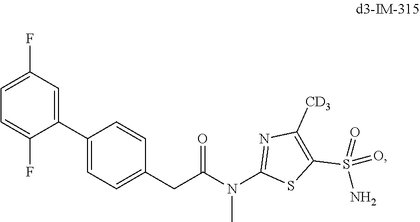 [SMILES: [2H]C([2H])([2H])c1nc(N(C)C(=O)Cc2ccc(-c3cc(F)ccc3F)cc2)sc1S(N)(=O)=O]

In some embodiments, the present disclosure is directed to a process for preparing these novel solid forms.

### DESCRIPTION OF FIGURES

FIG. 1 depicts a X-ray powder diffraction (XRPD) pattern of IM-250 free base Form I.

FIG. 2 depicts a combined thermogravimetric analysis (TGA) and differential scanning calorimeter (DSC) thermogram of IM-250 free base Form I.

FIG. 3 depicts a XRPD pattern of IM-250 free base Form III.

FIG. 4 depicts a combined TGA and DSC thermogram of IM-250 free base Form III.

FIG. 5 depicts a XRPD pattern of IM-250 HCl salt.

FIG. 6 depicts a combined TGA and DSC thermogram of IM-250 HCl salt.

FIG. 7 depicts a XRPD pattern of IM-250 HCl salt when crystallized from EtOH.

FIG. 8 depicts a XRPD pattern of IM-250 Napadisylate.

FIG. 9 depicts a combined TGA and DSC thermogram of IM-250 Napadisylate.

FIG. 10 depicts a XRPD pattern of IM-315.

FIG. 11 depicts a DSC thermogram of IM-315.

FIG. 12 depicts the diagram of the blood concentration over time of various solid forms of IM-250 in a mouse PK study.

FIG. 13 depicts the overlaid XRPD profiles (normalized scale) from IM-250 Free Base Form I of a non-stressed sample (bottom) and of samples stored for 2 weeks and 4 weeks at 40° C./75% RH and at 60° C.

FIG. 14 depicts a XRPD pattern of deuterated IM-250 free base (d3-IM-250).

FIG. 15 depicts a TGA thermogram of deuterated IM-250 free base (d3-IM-250).

FIG. 16 depicts a DSC thermogram of deuterated IM-250 free base (d3-IM-250).

FIG. 17 depicts a XRPD pattern of deuterated IM-250 HCl salt (d3-IM-250 HCl salt).

FIG. 18 depicts a combined TGA and DSC thermogram of deuterated IM-250 HCl salt (d3-IM-250 HCl salt).

### DETAILED DESCRIPTION

In the following description, certain specific details are set forth in order to provide a thorough understanding of various embodiments of the disclosure. However, one skilled in the art will understand that the disclosure may be practiced without these details. The description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

### Definitions

Unless the context requires otherwise, throughout the present specification and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment.

Furthermore, the particular features, structures, or characteristics may be combined in any suitable manner in one or more embodiments.

Embodiments that reference throughout this specification to “a crystalline form” include the crystalline, salt, co-crystal, hydrate and/or solvate of Formula (I) disclosed herein.

“Deuteration”, “deuterium labelled”, “deuterium substituted” or “deuterated” in the sense of the present disclosure means that one or more hydrogen atom(s) of the compound of Formula (I) is/are replaced by deuterium (²H, represented by “D”).

In some compounds of Formula (I), residue Y represents CD₃. It has surprisingly been found, that such deuterated aminothiazole compounds exhibit increased resistance to metabolism and thus be useful for increasing the half-life of a compound of Formula (I), compared to a respective undeuterated compound, when administered to a mammal, e.g. a human. See, for example, Foster in Trends Pharmacol. Sci. 1984:5; 524. Such deuterated aminothiazole compounds are synthesized by means well known in the art, for example by employing starting materials in which one or more hydrogens have been replaced by deuterium (see Experimental Section for details).

Deuterium labelled or substituted therapeutic compounds of the disclosure surprisingly turned out to have improved DMPK (drug metabolism and pharmacokinetics) properties, relating to absorption, distribution, metabolism and excretion (ADME). Substitution with deuterium turned out to afford certain therapeutic advantages resulting from greater metabolic stability, for example increased in vivo half-life, reduced dosage requirements and/or an improvement in therapeutic index.

The concentration of deuterium may be defined by an isotopic enrichment factor. In the compounds of this disclosure any atom not specifically designated as a particular isotope is meant to represent any stable or radioactive isotope of that atom. Unless otherwise stated, when a position is designated specifically as “H” or “hydrogen”, the position is understood to have hydrogen at its natural abundance isotopic composition (about 99.98% hydrogen). Accordingly, in the compounds of this disclosure any atom specifically designated as a deuterium (D) is meant to represent deuterium with an isotopic purity of at least 50%, preferably an isotopic purity of at least 95%, more preferably an isotopic purity of at least 99%.

The percentage of deuterium incorporation can be obtained by quantitative analysis using a number of conventional methods, such as mass spectroscopy (peak area) or by quantifying the remaining residual ¹H-NMR signals of the specific deuteration site compared to signals from internal standards or other, non-deuterated ¹H signals in the compound.

It will be recognized that some variation of natural isotopic abundance occurs in a synthesized compound depending upon the origin of chemical materials used in the synthesis. Thus, a preparation of non-deuterated analogs of compounds of the present invention will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Comp. Biochem. Physiol. 1998; 119A: 725.

The term “isotopic enrichment factor” at a particular position normally occupied by hydrogen refers to the ratio between the abundance of deuterium at the position and the natural abundance of deuterium at that position. By way of example, an isotopic enrichment factor of 3500 means that the amount of deuterium at the particular position is 3500-fold the natural abundance of deuterium, or that 52.5% of the compounds have deuterium at the particular position (i.e., 52.5% deuterium incorporation at the given position). The abundance of deuterium in the oceans of Earth is approximately one atom in 6500 hydrogen atoms (about 154 parts per million (ppm)). Deuterium thus accounts for approximately 0.015 percent (on a weight basis, 0.030 percent) of all naturally occurring hydrogen atoms in the oceans on Earth; the abundance changes slightly from one kind of natural water to another.

The deuterated compounds of this disclosure are preferably characterized by an isotopic enrichment factor of at least 6300, or by a deuteration degree of at least 95%. More preferably by an isotopic enrichment factor of at least 6500, or by a deuteration degree of at least 98%.

Any formula or structure given herein, is also intended to represent compounds comprising in addition further isotopically labeled atoms. Examples of additional isotopes that can be incorporated into compounds of the disclosure include further isotopes of hydrogen, as well as isotopes of carbon, nitrogen, oxygen and fluorine, such as, but not limited to ³H (tritium), ¹¹C, ¹³C, ¹⁴C, ¹⁵N, ¹⁸F and ³⁵S. The disclosure further comprises various isotopically labeled compounds into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or radioactive treatment of patients. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, and/or emulsifier, or a combination of one or more of the above which has been approved by the United States Food and Drug Administration (FDA), European Medicines Agency (EMA) or other national counterparts as being acceptable for use in humans or domestic animals.

A “pharmaceutical composition” refers to a formulation of a compound of the disclosure (e.g. a compound of Formula (I)) and a medium (administration form) generally accepted in the art for the delivery of the biologically active compound to mammals, e.g. humans. Such a medium includes all pharmaceutically acceptable excipients therefor.

The term “effective amount” is meant to include the amount of a compound that, when administered, is sufficient to prevent development of, or alleviate to some extent, an infection or one or more of the symptoms of a disorder, disease, or condition being treated. The term “effective amount” also refers to the amount of a compound that is sufficient to elicit the biological or medical response of a cell, tissue, system, animal, or human, which is being sought by a researcher, veterinarian, medical doctor, or clinician.

“Prevention” or “preventing” or “prophylaxis” means any treatment of an infection, disease or condition that causes the clinical symptoms of the disease or condition not to develop.

Compounds may, in some embodiments, be administered to a subject (including a human) who is at risk or has a family history of an infection, disease or condition.

“Treating” and “treatment” of a disease include the following:
(1) preventing or reducing the risk of developing the disease, i.e. causing the clinical symptoms of the disease not to develop in a subject that may be exposed to or predisposed to the disease but does not yet experience or display symptoms of the disease,
(2) inhibiting the disease, i.e. arresting or reducing the development of the disease or its clinical symptoms,
(3) relieving (healing) the disease, i.e. causing regression of the disease or its clinical symptoms, and
(4) ameliorating or alleviating the symptoms or impairments caused by the disease.

The terms “subject” or “patient” refer to an animal, such as a mammal (including a human), that has been or will be the object of treatment, observation or experiment. The methods described herein may be useful in human therapy and/or veterinary applications. In some embodiments, the subject is a mammal (or the patient). In some embodiments the subject (or the patient) is human, domestic animals (e.g. dogs and cats), farm animals (e.g. cattle, horses, sheep, goats, and pigs) and/or laboratory animals (e.g. mice, rats, hamsters, guinea pigs, pigs, rabbits, dogs, and monkeys). In some embodiments, the subject (or the patient) is a human. “Human (or patient) in need thereof” refers to a human who may have or is suspect to have an infection or disease or conditions that would benefit from certain treatment; for example, being treated with the compounds disclosed herein according to the present application.

Reference to “about” a value or parameter herein includes (and describes) embodiments that are directed to that value or parameter per se. For example, description referring to “about x” includes description of “x”. Also, the singular forms “a” and “the” include plural references unless the context clearly dictates otherwise. Thus, e.g. reference to “the compound” includes a plurality of such compounds and reference to “the assay” includes reference to one or more assays and equivalents thereof known to those skilled in the art.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “substantially as shown in” when referring, for example, to an XRPD pattern, a DSC thermogram or a TGA thermogram includes a pattern, thermogram or spectrum that is not necessarily identical to those depicted herein, but that falls within the limits of experimental error or deviations when considered by one of ordinary skill in the art.

The term “pharmaceutically acceptable salts” refers to salts prepared from pharmaceutically acceptable non-toxic bases or acids, including inorganic bases or acids and organic bases or acids. In case the compounds of the present disclosure contain one or more acidic or basic groups, the disclosure also comprises their corresponding pharmaceutically or toxicologically acceptable salts, in particular their pharmaceutically utilizable salts. Thus, the compounds of the present disclosure which contain acidic groups can be present on these groups and can be used according to the disclosure, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The compounds of the present disclosure which contain one or more basic groups, i.e. groups which can be protonated, can be present and can be used according to the disclosure in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the present disclosure simultaneously contain acidic and basic groups in the molecule, the disclosure also includes, in addition to the salt forms mentioned, inner salts or betaines (zwitterions).

The respective salts can be obtained by customary methods which are known to the person skilled in the art like, for example, by contacting these with an organic or inorganic acid or base in a solvent or dispersant, or by anion exchange or cation exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts.

Generally, the salt formation of the compounds according to Formula (I) can be carried out by conventional crystallization methods. Preferably the crystallization is carried out by contacting the compounds of the Formula (I) with a water-miscible solvent or solvent mixture and adding the selected acid or base for forming the respective salt. The resulting crystallized salts are isolated by usual methods, comprising e.g. filtration, washing and drying.

Further the compounds of the present disclosure may be present in the form of solvates, such as those which include as solvate water, or pharmaceutically acceptable solvates, such as alcohols, in particular ethanol. A “solvate” is formed by the interaction of a solvent and a compound. When the solvent is water, the “solvate” is a “hydrate”. It is understood, that also a salt of the present disclosure can include a solvate.

Suitable solvents for salt- and solvent formation of the compounds according to Formula (I) as defined herein comprise: acetonitrile, dichloromethane (DCM), alcohols, such as especially methanol, ethanol, 2-propanol (iso-propanol), aldehydes, ketones, especially acetone, ethers, e.g. tetrahydrofuran (THF) or dioxane, esters, e.g. ethyl acetate, or alkanes, such as especially pentane, hexane, heptane or cyclohexane and water, and mixtures thereof.

In certain embodiments, provided are optical isomers, racemates or other mixtures thereof of the compounds described herein or a pharmaceutically acceptable salt or a mixture thereof. If desired, isomers can be separated by methods well known in the art, e.g. by liquid chromatography. In those situations, the single enantiomer or diastereomer, i.e. optically active form, can be obtained by asymmetric synthesis or by resolution. Resolution can be accomplished, for example, by conventional methods such as crystallization in the presence of a resolving agent, or chromatography, using for example, a chiral high-pressure liquid chromatography (HPLC) column or a chiral supercritical fluid chromatography (SFC) column.

A “stereoisomer” refers to a compound made up of the same atoms bonded by the same bonds but having different three-dimensional structures, which are not interchangeable. The term “enantiomer” means each individual optically active form of a compound of the invention, having an optical purity or enantiomeric excess (as determined by methods standard in the art) of at least 80% (i.e. at least 90% of one enantiomer and at most 10% of the other enantiomer), preferably at least 90% and more preferably at least 98%.

The compounds disclosed herein and their pharmaceutically acceptable salts may include an asymmetric center and may thus give rise to enantiomers, diastereomers and other stereoisomeric forms that may be defined, in terms of absolute stereochemistry, as I- or(S)-. The present invention is meant to include all such possible isomers, as well as their racemic and optically pure forms. Optically active (+) and (−) or (R)- and (S)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, HPLC or SFC.

### Solid Forms of Compounds of Formula (I)

Solid forms of compounds of Formula (I), including crystalline forms and substantially pure forms, may provide the advantage of bioavailability and stability, suitable for use as an active ingredient in a pharmaceutical composition. Surprisingly, IM-250 HCl salt, for example, exhibits advantageous physical properties such as good physical and chemical stability, good aqueous solubility and good bioavailability, while being non-hygroscopic. Variations in the crystal structure of a pharmaceutical drug substance or active ingredient may affect the dissolution rate (which may affect bioavailability etc.), manufacturability (e.g. ease of handling, ability to consistently prepare doses of known strength) and stability (e.g. thermal stability, shelf life etc.) of a pharmaceutical drug product or active ingredient. Such variations may affect the preparation or formulation of pharmaceutical compositions in different dosage or delivery forms, such as solutions or solid oral dosage form including tablets and capsules. Compared to other forms such as non-crystalline or amorphous forms, specific crystalline forms may provide desired or suitable hygroscopicity, particle size control, improvement of dissolution rate, solubility, purity, physical and chemical stability, manufacturability, yield and/or process control. Thus, solid (crystalline) forms of the compound of Formula (I) may provide advantages such as improving: the manufacturing process of the compound, the stability or storability of a drug product form of the compound, the stability or storability of a drug substance of the compound and/or the bioavailability and/or stability of the compound as an active agent.

The use of certain solvents and/or processes have been found to produce different solid forms of the compounds of Formula (I) described herein which may exhibit one or more favorable characteristics described above. The processes for the preparation of the solid forms described herein and characterization of these solid forms are described in detail below.

In particular embodiments, novel solid forms, such as crystalline forms of compounds of Formula (I) are disclosed.

The invention relates in particular to the following embodiments:

In a preferred embodiment in combination with any of the above or below embodiments X is selected from

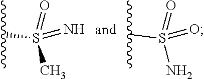

In a more preferred embodiment in combination with any of the above or below embodiments X is

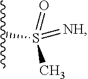 [SMILES: *[S@@](C)(=N)=O]

In a preferred embodiment in combination with any of the above or below embodiments Y is selected from CH₃ and CD₃.

In a more preferred embodiment in combination with any of the above or below embodiments Y is CH₃.

In a further preferred embodiment in combination with any of the above or below embodiments X is selected from

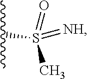 [SMILES: *[S@@](C)(=N)=O]

while Y is CH₃ or CD₃.

In a further embodiment in combination with any of the above or below embodiments X is selected from

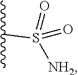 [SMILES: *S(N)(=O)=O]

while Y is CD₃, therewith excluding the compound with the general formula

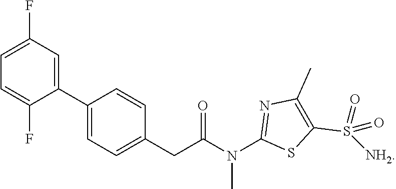

A preferred embodiment in combination with any of the above or below embodiments relates to a solid (crystalline) form of a compound according Formula (I) having the structure

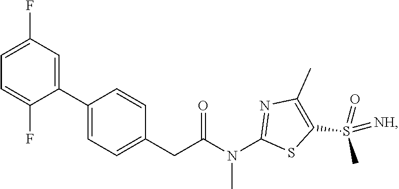 [SMILES: Cc1nc(N(C)C(=O)Cc2ccc(-c3cc(F)ccc3F)cc2)sc1[S@@](C)(=N)=O]

or a pharmaceutically acceptable salt, co-crystal, hydrate or solvate thereof.

Another preferred embodiment in combination with any of the above or below embodiments relates to a solid (crystalline) form of a compound according Formula (I) having the structure

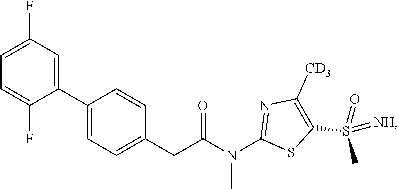 [SMILES: [2H]C([2H])([2H])c1nc(N(C)C(=O)Cc2ccc(-c3cc(F)ccc3F)cc2)sc1[S@@](C)(=N)=O]

or a pharmaceutically acceptable salt, co-crystal, hydrate or solvate thereof.

Another preferred embodiment in combination with any of the above or below embodiments relates to a solid (crystalline) form of a compound according Formula (I) having the structure

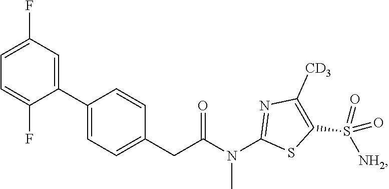

or a pharmaceutically acceptable co-crystal, hydrate or solvate thereof.

Another preferred embodiment in combination with any of the above or below embodiments relates to a solid (crystalline) form of a compound according Formula (I) having the structure

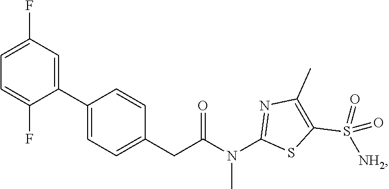 [SMILES: Cc1nc(N(C)C(=O)Cc2ccc(-c3cc(F)ccc3F)cc2)sc1S(N)(=O)=O]

or a pharmaceutically acceptable co-crystal, hydrate or solvate thereof, which are preferably further characterized by having a melting point of 197° C. (±5° C.) and/or by being a white solid.

### IM-250 Free Base

One embodiment of the invention relates to a solid (crystalline) compound according to Formula (I) having the structure

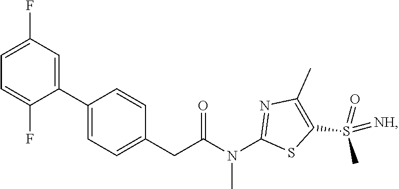 [SMILES: Cc1nc(N(C)C(=O)Cc2ccc(-c3cc(F)ccc3F)cc2)sc1[S@@](C)(=N)=O]

in the form of the free base.

### IM-250 Free Base Form I

In some embodiments such IM-250 Free Base Form is present in a solid crystalline form, IM-250 Free base Form I, with the structure

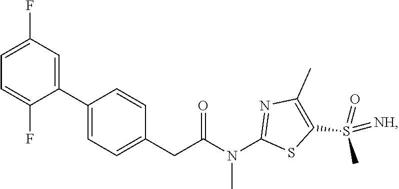 [SMILES: Cc1nc(N(C)C(=O)Cc2ccc(-c3cc(F)ccc3F)cc2)sc1[S@@](C)(=N)=O]

wherein this solid IM-250 Free base Form I is characterized by an X-ray powder diffraction pattern (XRPD) comprising (characteristic peaks) degree 2θ-reflections (±0.2 degrees 2θ) at 9.2, 13.7 and 18.7 degrees.

In some embodiments, IM-250 Free base Form I is characterized by an XRPD pattern comprising degree 2θ-reflections (±0.2 degrees 2θ) at 9.2, 13.7 and 18.7 degrees and one, two or three of the degree 2θ-reflections (±0.2 degrees 2θ) at 14.4, 24.0 and 27.3 degrees.

In some embodiments, IM-250 Free base Form I is characterized by an XRPD pattern comprising degree 2θ-reflections (±0.2 degrees 2θ) at 9.2, 13.7, 14.4, 18.7, 24.0 and 27.3 degrees.

In some embodiments, IM-250 Free base Form I is characterized by an XRPD pattern comprising at least 4 of the following (characteristic) peaks: 9.2, 13.7, 14.4, 18.7, 24.0 and 27.3 degrees 2θ (±0.2 degrees 2θ).

All values as determined on a diffractometer using Cu-Kα radiation at a wavelength of 1.54 Å.

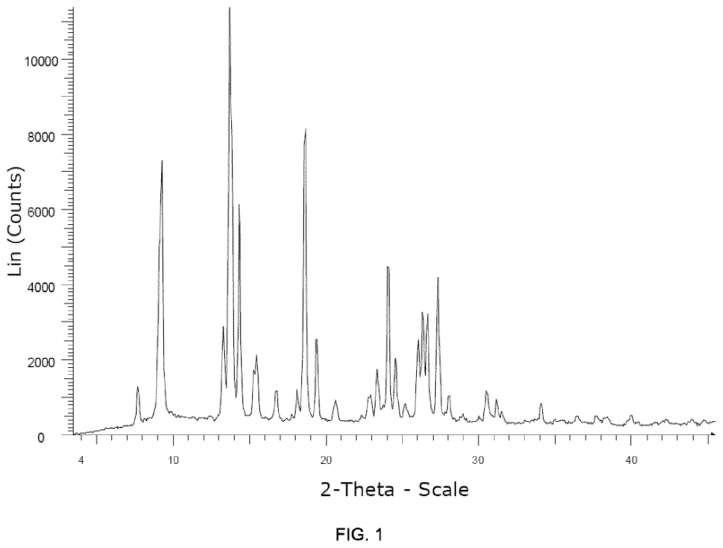

In some embodiments, crystalline IM-250 Free base Form I has an XRPD pattern displaying at least two, at least three, at least four, at least five, at least six, at least seven, at least eight, or at least nine of the degree 2θ-reflections with the greatest intensity as the XRPD pattern substantially as shown in FIG. 1.

More preferably, such solid free base form, IM-250 Free base Form I, exhibits an X-ray powder diffraction (XRPD) pattern substantially as shown in FIG. 1.

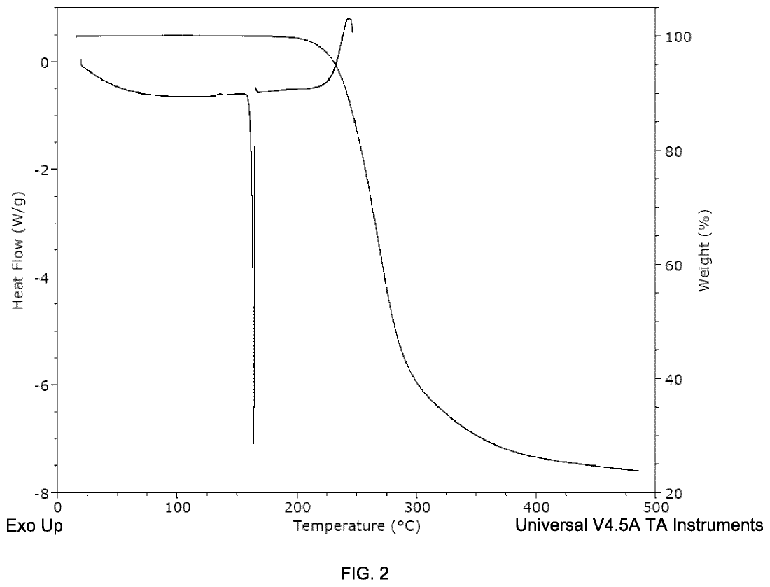

Further, such IM-250 Free base Form I may exhibit a differential scanning calorimetry (DSC) thermogram substantially as shown in FIG. 2.

Further, such IM-250 Free base Form I may exhibit a thermogravimetric analysis (TGA) thermogram substantially as shown in FIG. 2.

In some embodiments of crystalline IM-250 Free base Form I, at least one, at least two, or all of the following (a)-(c) apply: (a) crystalline IM-250 Free base Form I has an XRPD pattern substantially as shown in FIG. 1; (b) crystalline IM-250 Free base Form I has a DSC thermogram substantially as shown in FIG. 2; (c) crystalline IM-250 Free base Form I has a TGA thermogram substantially as shown in FIG. 2.

In some embodiments, crystalline IM-250 Free base Form I has at least one, at least two, or at least three of the following properties:
(a) an XRPD pattern substantially as shown in FIG. 1
(b) a DSC thermogram substantially as shown in FIG. 2
(c) a TGA thermogram substantially as shown in FIG. 2.

In some embodiments, IM-250 Free base Form I has a differential scanning calorimetry thermogram comprising an endotherm with an onset at about 163° C.

Accordingly, a solid IM-250 Free base Form I may further be characterized by having a melting point of 164 to 165° C. (±5° C.).

### IM-250 Free Base Form III

In some embodiments such IM-250 Free Base Form is present in a solid crystalline form IM-250 Free base Form Ill with the structure

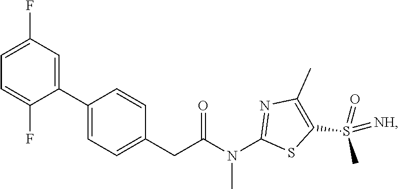 [SMILES: Cc1nc(N(C)C(=O)Cc2ccc(-c3cc(F)ccc3F)cc2)sc1[S@@](C)(=N)=O]

wherein this solid IM-250 Free base Form Ill is characterized by an XRPD pattern comprising (characteristic peaks) degree 2θ-reflections (±0.2 degrees 2θ) at 9.7, 12.3 and 15.6 degrees.

In some embodiments, IM-250 Free base Form III is characterized by an XRPD pattern comprising degree 2θ-reflections (±0.2 degrees 2θ) at 9.7, 12.3 and 15.6 degrees and one, two or three of the degree 2θ-reflections (±0.2 degrees 2θ) at 12.9, 22.7 and 23.8 degrees.

In some embodiments, IM-250 Free base Form III is characterized by an XRPD pattern comprising degree 2θ-reflections (±0.2 degrees 2θ) at 9.7, 12.3, 12.9, 15.6, 22.7 and 23.8 degrees.

In some embodiments, IM-250 Free base Form Ill is characterized by an XRPD pattern comprising at least 4 of the following (characteristic) peaks: 9.7, 12.3, 12.9, 15.6, 22.7 and 23.8 degrees 2θ (±0.2 degrees 2θ).

All values determined on a diffractometer using Cu-Kα radiation at a wavelength of 1.54 Å.

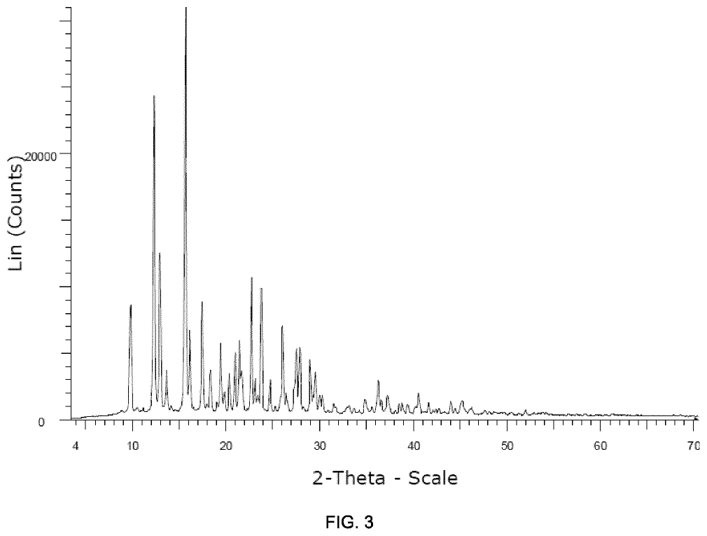

In some embodiments, crystalline IM-250 Free base Form Ill has an XRPD pattern displaying at least two, at least three, at least four, at least five, at least six, at least seven, at least eight, or at least nine of the degree 2θ-reflections with the greatest intensity as the XRPD pattern substantially as shown in FIG. 3.

More preferably, such solid free base form, IM-250 Free base Form III, exhibits an X-ray powder diffraction (XRPD) pattern substantially as shown in FIG. 3.

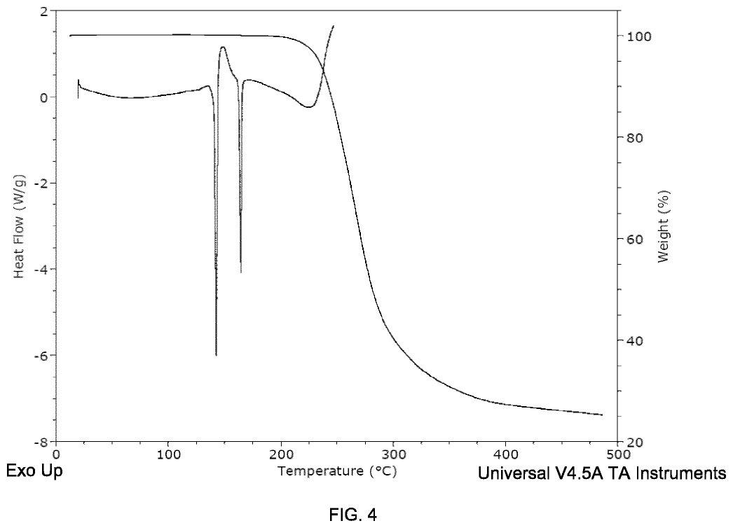

Further, such IM-250 Free base Form Ill may exhibit a differential scanning calorimetry (DSC) thermogram substantially as shown in FIG. 4.

Further, such IM-250 Free base Form Ill may exhibit a thermogravimetric analysis (TGA) thermogram substantially as shown in FIG. 4.

In some embodiments of crystalline IM-250 Free base Form III, at least one, at least two, or all of the following (a)-(c) apply: (a) crystalline IM-250 Free base Form Ill has an XRPD pattern substantially as shown in FIG. 3; (b) crystalline IM-250 Free base Form Ill has a DSC thermogram substantially as shown in FIG. 4; (c) crystalline IM-250 Free base Form Ill has a TGA thermogram substantially as shown in FIG. 4.

In some embodiments, crystalline IM-250 Free base Form Ill has at least one, at least two, or at least three of the following properties:
(a) an XRPD pattern substantially as shown in FIG. 3
(b) a DSC thermogram substantially as shown in FIG. 4
(c) a TGA thermogram substantially as shown in FIG. 4.

In some embodiments, IM-250 Free base Form Ill has a differential scanning calorimetry thermogram comprising an endotherm with an onset at about 141° C.

Accordingly, a solid IM-250 Free base Form Ill may further be characterized by having a melting point of 143° C. (±5° C.).

Deuterated IM-250 Free Base (d3-IM-250 Free Base)

A further embodiment of the invention relates to a solid (crystalline) compound according to Formula (I), which is deuterated IM-250 free base (d3-IM-250 free base), with the structure

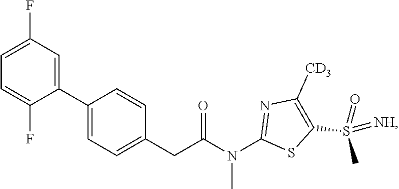 [SMILES: [2H]C([2H])([2H])c1nc(N(C)C(=O)Cc2ccc(-c3cc(F)ccc3F)cc2)sc1[S@@](C)(=N)=O]

wherein this solid deuterated IM-250 free base (d3-IM-250 free base) is characterized by an X-ray powder diffraction pattern (XRPD) comprising (characteristic peaks) degree 2θ-reflections (±0.2 degrees 2θ) at 9.3, 13.7 and 18.6 degrees.

In some embodiments, deuterated IM-250 free base (d3-IM-250 free base) is characterized by an XRPD pattern comprising degree 2θ-reflections (±0.2 degrees 2θ) at 9.3, 13.7 and 18.6 degrees and one, two, three or four of the degree 2θ-reflections (±0.2 degrees 2θ) at 14.4, 15.3, 15.5 and 24.1 degrees.

In some embodiments, deuterated IM-250 free base (d3-IM-250 free base) is characterized by an XRPD pattern comprising degree 2θ-reflections (±0.2 degrees 2θ) at 9.3, 13.7, 14.4, 15.3, 15.5, 18.6 and 24.1 degrees.

In some embodiments, deuterated IM-250 free base (d3-IM-250 free base) is characterized by an XRPD pattern comprising at least 4 of the following (characteristic) peaks: 9.3, 13.7, 14.4, 15.3, 15.5, 18.6 and 24.1 degrees 2θ (±0.2 degrees 2θ).

All values as determined on a diffractometer using Cu-Kα radiation at a wavelength of 1.54 Å.

In some embodiments, crystalline deuterated IM-250 free base (d3-IM-250 free base) has an XRPD pattern displaying at least two, at least three, at least four, at least five, at least six, at least seven, at least eight, or at least nine of the degree 2θ-reflections with the greatest intensity as the XRPD pattern substantially as shown in FIG. 14.

More preferably, such solid free base form, deuterated IM-250 free base (d3-IM-250 free base), exhibits an X-ray powder diffraction (XRPD) pattern substantially as shown in FIG. 14.

Further, such deuterated IM-250 free base (d3-IM-250 free base) may exhibit a differential scanning calorimetry (DSC) thermogram substantially as shown in FIG. 16.

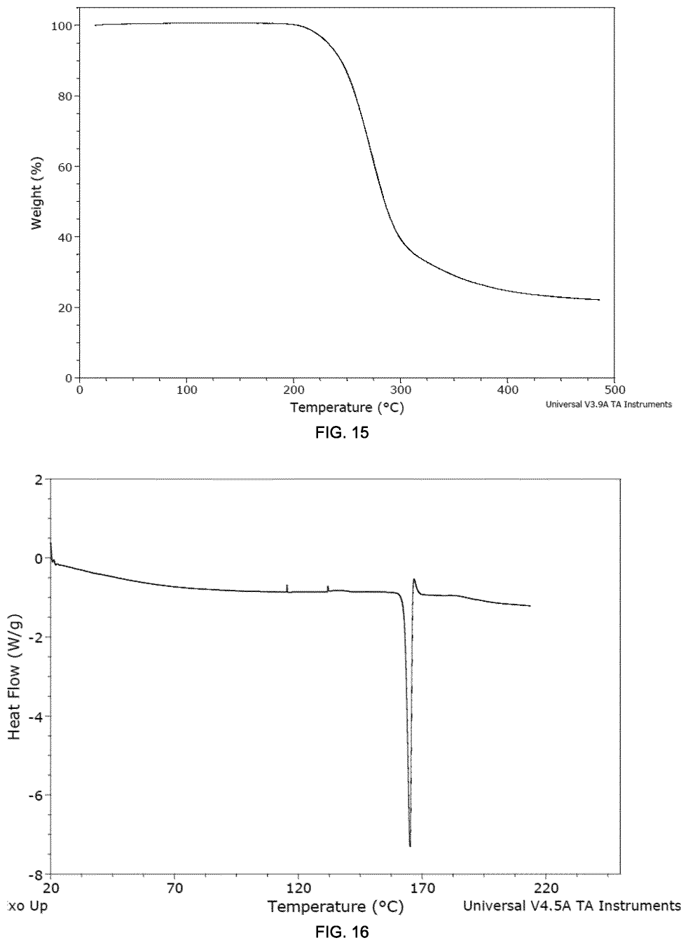

Further, such deuterated IM-250 free base (d3-IM-250 free base) may exhibit a thermogravimetric analysis (TGA) thermogram substantially as shown in FIG. 15.

In some embodiments of crystalline deuterated IM-250 free base (d3-IM-250 free base), at least one, at least two, or all of the following (a)-(c) apply: (a) crystalline deuterated IM-250 free base (d3-IM-250 free base) has an XRPD pattern substantially as shown in FIG. 14; (b) crystalline deuterated IM-250 free base (d3-IM-250 free base) has a DSC thermogram substantially as shown in FIG. 16; (c) crystalline deuterated IM-250 free base (d3-IM-250 free base) has a TGA thermogram substantially as shown in FIG. 15.

In some embodiments, crystalline deuterated IM-250 free base (d3-IM-250 free base) has at least one, at least two, or at least three of the following properties:
(a) an XRPD pattern substantially as shown in FIG. 14
(b) a DSC thermogram substantially as shown in FIG. 16
(c) a TGA thermogram substantially as shown in FIG. 15.

In some embodiments, deuterated IM-250 free base (d3-IM-250 free base) has a differential scanning calorimetry thermogram comprising an endotherm with an onset at about 163° C.

Accordingly, a solid deuterated IM-250 free base (d3-IM-250 free base) may further be characterized by having a melting point of 163 to 165° C. (±5° C.).

### Selected Salt Forms of Compounds of the Invention

A further embodiment of the invention relates to selected salt forms of the compounds according to Formula (I), preferably compounds of Formula (I) wherein
X is

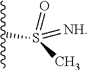 [SMILES: *[S@@](C)(=N)=O]

Particularly preferred are HCl salts and napadisylate salts of the compounds of the present invention, such as in particular HCl salts and napadisylate salts of compounds of Formula (I), wherein HCl salts are most preferred, and wherein
X is

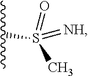 [SMILES: *[S@@](C)(=N)=O]

and
Y is selected from CH₃ and CD₃;
and a hydrate or solvate thereof.

### IM-250 HCl Salt

A further embodiment of the invention relates to a HCl salt of compound IM-250, which is IM-250 HCl salt with the structure

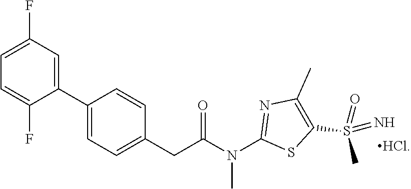

In one embodiment of the invention, such IM-250 HCl salt is characterized by an XRPD pattern comprising (characteristic peaks) degree 2θ-reflections (±0.3 degrees 2θ) at 13.7, 17.7 and 22.8 degrees.

In some embodiments, IM-250 HCl salt is characterized by an XRPD pattern comprising degree 2θ-reflections (±0.3 degrees 2θ) at 13.7, 17.7 and 22.8 degrees and one, two or three of the degree 2θ-reflections (±0.3 degrees 2θ) at 17.0, 19.8 and 21.8 degrees.

In some embodiments, IM-250 HCl salt is characterized by an XRPD pattern comprising degree 2θ-reflections (±0.3 degrees 2θ) at 13.7, 17.0, 17.7, 19.8, 21.8 and 22.8 degrees.

In some embodiments, IM-250 HCl salt is characterized by an XRPD pattern comprising at least 4 of the following peaks: 13.7, 17.0, 17.7, 19.8, 21.8 and 22.8 degrees 2θ (±0.3 degrees 2θ).

All values determined on a diffractometer using Cu-Kα radiation at a wavelength of 1.54 Å.

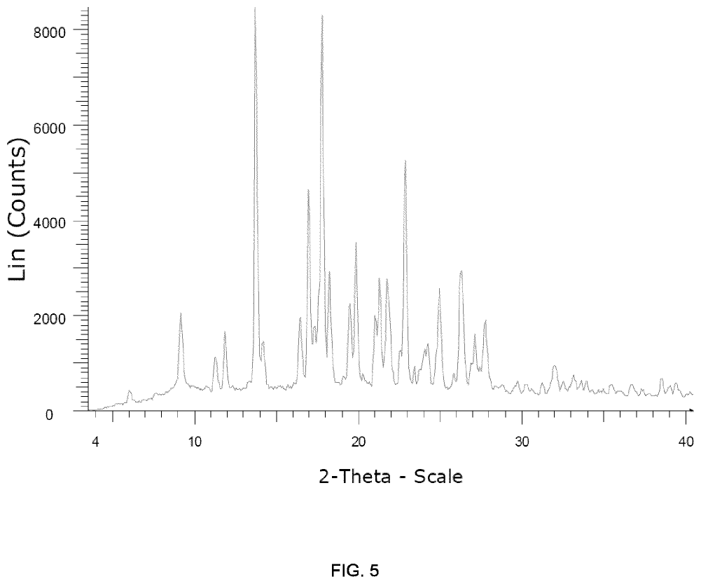

In some embodiments, crystalline IM-250 HCl salt has an XRPD pattern displaying at least two, at least three, at least four, at least five, at least six, at least seven, at least eight, or at least nine of the degree 2θ-reflections with the greatest intensity as the XRPD pattern substantially as shown in FIG. 5.

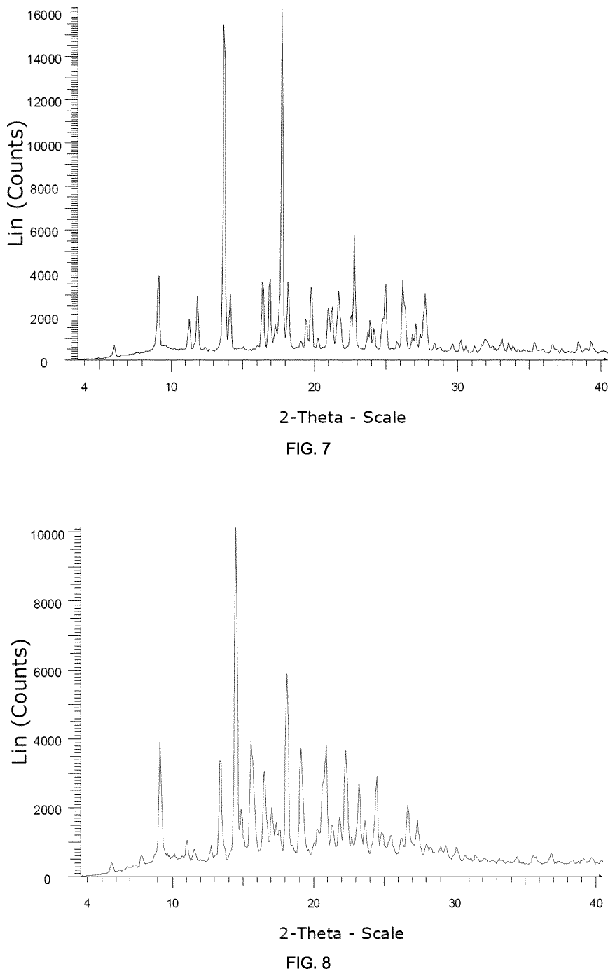

In some embodiments, crystalline IM-250 HCl salt has an XRPD pattern displaying at least two, at least three, at least four, at least five, at least six, at least seven, at least eight, or at least nine of the degree 2θ-reflections with the greatest intensity as the XRPD pattern substantially as shown in FIG. 7.

In some embodiments, such solid HCl salt form IM-250 HCl salt exhibits an X-ray powder diffraction (XRPD) pattern substantially as shown in FIG. 5.

In some embodiments, such solid HCl salt form IM-250 HCl salt exhibits an X-ray powder diffraction (XRPD) pattern substantially as shown in FIG. 7.

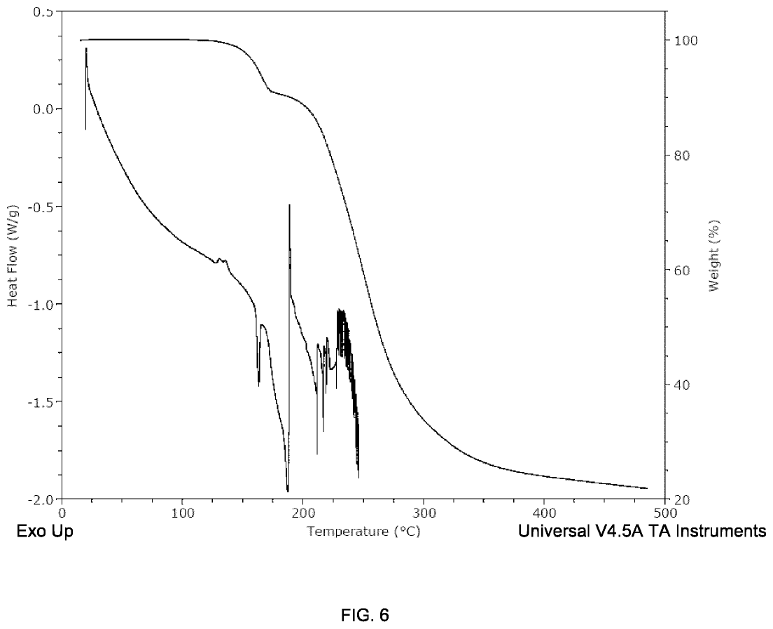

Further, such IM-250 HCl salt may exhibit a differential scanning calorimetry (DSC) thermogram substantially as shown in FIG. 6.

Further, such IM-250 HCl salt may exhibit a thermogravimetric analysis (TGA) thermogram substantially as shown in FIG. 6.

In some embodiments of crystalline IM-250 HCl salt, at least one, at least two, or all of the following (a)-(c) apply: (a) crystalline IM-250 HCl salt has an XRPD pattern substantially as shown in FIG. 5; (b) crystalline IM-250 HCl salt has a DSC thermogram substantially as shown in FIG. 6; (c) crystalline IM-250 HCl salt has a TGA thermogram substantially as shown in FIG. 6.

In some embodiments, crystalline IM-250 HCl salt has at least one, at least two, or at least three of the following properties:
(a) an XRPD pattern substantially as shown in FIG. 5
(b) a DSC thermogram substantially as shown in FIG. 6
(c) a TGA thermogram substantially as shown in FIG. 6.

In some embodiments of crystalline IM-250 HCl salt, at least one, at least two, or all of the following (a)-(c) apply: (a) crystalline IM-250 HCl salt has an XRPD pattern substantially as shown in FIG. 7; (b) crystalline IM-250 HCl salt has a DSC thermogram substantially as shown in FIG. 6; (c) crystalline IM-250 HCl salt has a TGA thermogram substantially as shown in FIG. 6.

In some embodiments, crystalline IM-250 HCl salt has at least one, at least two, or at least three of the following properties:
(a) an XRPD pattern substantially as shown in FIG. 7
(b) a DSC thermogram substantially as shown in FIG. 6
(c) a TGA thermogram substantially as shown in FIG. 6.

In some embodiments, IM-250 HCl salt has a thermogravimetric analysis thermogram revealing a mass loss of about 9.8% upon heating with an onset/endset temperature of about 151/170° C.

In some embodiments, IM-250 HCl salt has a thermogravimetric analysis thermogram revealing a onset temperature of decomposition of about 221° C.

In some embodiments, provided herein is IM-250 HCl salt with structure

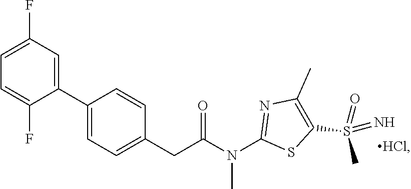

wherein hydrochloride and (S)-2-(2′,5′-difluoro-[1,1′-biphenyl]-4-yl)-N-methyl-N-(4-methyl-5-(S-methylsulfonimidoyl)thiazol-2-yl)acetamide are in a 1 to 1±0.2 molar ratio.

The HCl salt, IM-250 HCl salt, surprisingly turned out to exhibit several advantages with respect to chemical and physical stability, (lack of) hygroscopicity and improved bioavailability, while other tested salts, as shown in the Examples below, were less advantageous. Accordingly, a compound, IM-250 HCl salt, is a particularly preferred embodiment of the present invention.

### Deuterated IM-250 HCl Salt-d3-IM-250 HCl Salt

A further embodiment of the invention relates to a HCl salt of the corresponding deuterated compound of compound IM-250 HCl salt (d3-IM-250 HCl salt) with the structure

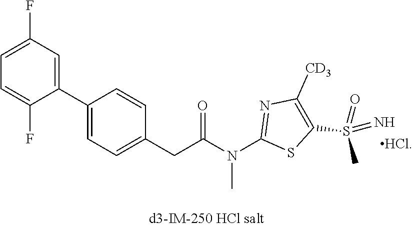

In one embodiment of the invention, such deuterated IM-250 HCl salt (d3-IM-250 HCl salt) is characterized by an XRPD pattern comprising (characteristic peaks) degree 2θ-reflections (±0.3 degrees 2θ) at 13.8, 17.8 and 21.8 degrees.

In some embodiments, deuterated IM-250 HCl salt (d3-IM-250 HCl salt) is characterized by an XRPD pattern comprising degree 2θ-reflections (±0.3 degrees 2θ) at 13.8, 17.8 and 21.8 degrees and one, two, three, four or five of the degree 2θ-reflections (±0.3 degrees 2θ) at 11.3, 11.9, 19.8, 21.0 and 21.3 degrees.

In some embodiments, deuterated IM-250 HCl salt (d3-IM-250 HCl salt) is characterized by an XRPD pattern comprising degree 2θ-reflections (±0.3 degrees 2θ) at 11.3, 11.9, 13.8, 17.8, 19.8, 21.0, 21.3 and 21.8 degrees.

In some embodiments, deuterated IM-250 HCl salt (d3-IM-250 HCl salt) is characterized by an XRPD pattern comprising at least 4 of the following peaks: 11.3, 11.9, 13.8, 17.8, 19.8, 21.0, 21.3 and 21.8 degrees 2θ (±0.3 degrees 2θ).

All values determined on a diffractometer using Cu-Kα radiation at a wavelength of 1.54 Å.

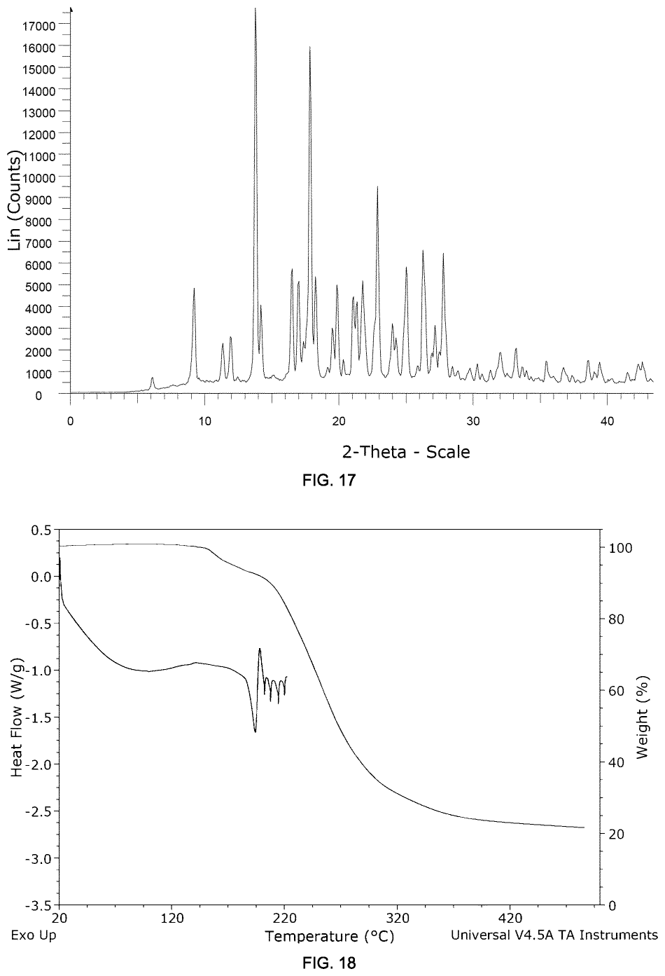

In some embodiments, crystalline deuterated IM-250 HCl salt (d3-IM-250 HCl salt) has an XRPD pattern displaying at least two, at least three, at least four, at least five, at least six, at least seven, at least eight, or at least nine of the degree 2θ-reflections with the greatest intensity as the XRPD pattern substantially as shown in FIG. 17.

In some embodiments, such solid HCl salt form deuterated IM-250 HCl salt (d3-IM-250 HCl salt) exhibits an X-ray powder diffraction (XRPD) pattern substantially as shown in FIG. 17.

Further, such deuterated IM-250 HCl salt (d3-IM-250 HCl salt) may exhibit a differential scanning calorimetry (DSC) thermogram substantially as shown in FIG. 18.

Further, such deuterated IM-250 HCl salt (d3-IM-250 HCl salt) may exhibit a thermogravimetric analysis (TGA) thermogram substantially as shown in FIG. 18.

In some embodiments of crystalline deuterated IM-250 HCl salt (d3-IM-250 HCl salt), at least one, at least two, or all of the following (a)-(c) apply: (a) crystalline deuterated IM-250 HCl salt (d3-IM-250 HCl salt) has an XRPD pattern substantially as shown in FIG. 17; (b) crystalline deuterated IM-250 HCl salt (d3-IM-250 HCl salt) has a DSC thermogram substantially as shown in FIG. 18; (c) crystalline deuterated IM-250 HCl salt (d3-IM-250 HCl salt) has a TGA thermogram substantially as shown in FIG. 18.

In some embodiments, crystalline deuterated IM-250 HCl salt (d3-IM-250 HCl salt) has at least one, at least two, or at least three of the following properties:
(a) an XRPD pattern substantially as shown in FIG. 17
(b) a DSC thermogram substantially as shown in FIG. 18
(c) a TGA thermogram substantially as shown in FIG. 18.

In some embodiments, deuterated IM-250 HCl salt (d3-IM-250 HCl salt) has a thermogravimetric analysis thermogram revealing a mass loss of about 7.8% upon heating with an onset/endset temperature of about 149/167° C.

In some embodiments, deuterated IM-250 HCl salt (d3-IM-250 HCl salt) has a thermogravimetric analysis thermogram revealing a onset temperature of decomposition of about 225° C.

In some embodiments, deuterated IM-250 HCl salt (d3-IM-250 HCl salt) has a differential scanning calorimetry thermogram comprising an endotherm with an onset at about 188° C.

Accordingly, a solid deuterated IM-250 HCl salt (d3-IM-250 HCl salt) may further be characterized by having a melting point of 188 to 194° C. (±5° C.).

In some embodiments, provided herein is deuterated IM-250 HCl salt (d3-IM-250 HCl salt) with structure

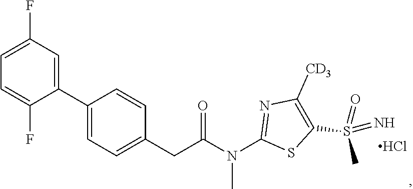 [SMILES: [2H]C([2H])([2H])c1nc(N(C)C(=O)Cc2ccc(-c3cc(F)ccc3F)cc2)sc1[S@@](C)(=N)=O ; Cl]

wherein hydrochloride and (S)-2-(2′,5′-difluoro-[1,1′-biphenyl]-4-yl)-N-methyl-N-(4-(methyl-d₃)-5-(S-methylsulfonimidoyl)thiazol-2-yl)acetamide are in a 1 to 1±0.2 molar ratio.

The deuterated HCl salt, deuterated IM-250 HCl salt (d3-IM-250 HCl salt), surprisingly turned out to exhibit several advantages with respect to chemical and physical stability, (lack of) hygroscopicity and improved bioavailability, while other tested salts were less advantageous. Accordingly, crystalline deuterated IM-250 HCl salt (d3-IM-250 HCl salt) is a particularly preferred embodiment of the present invention.

### IM-250 Napadisylate Salt

A further embodiment of the invention relates to a naphthalenedisulfonic acid salt of a compound IM-250, which is IM-250 Napadisylate with the structure

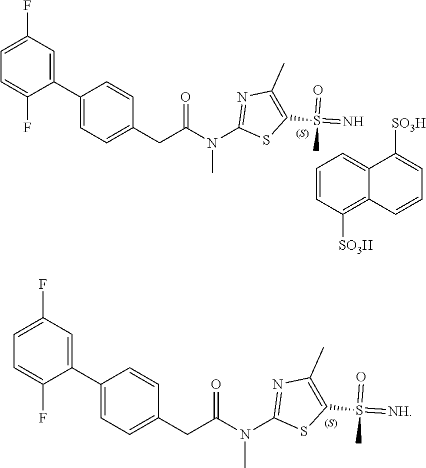

In one embodiment of the invention, such IM-250 Napadisylate is characterized by having an XRPD pattern comprising (characteristic peaks) degree 2θ-reflections (±0.2 degrees 2θ) at 9.1, 14.5 and 18.1 degrees.

In some embodiments, IM-250 Napadisylate is characterized by an XRPD pattern comprising degree 2θ-reflections (±0.2 degrees 2θ) at 9.1, 14.5 and 18.1 degrees and one, two or three of the degree 2θ-reflections (±0.2 degrees 2θ) at 15.6, 19.1 and 20.9 degrees.

In some embodiments, IM-250 Napadisylate is characterized by an XRPD pattern comprising degree 2θ-reflections (±0.2 degrees 2θ) at 9.1, 14.5, 15.6, 18.1, 19.1 and 20.9 degrees.

In some embodiments, IM-250 Napadisylate is characterized by an XRPD pattern comprising at least 4 of the following peaks: 9.1, 14.5, 15.6, 18.1, 19.1 and 20.9 degrees 2θ (±0.2 degrees 2θ).

All values determined on a diffractometer using Cu-Kα radiation at a wavelength of 1.54 Å.

In some embodiments, crystalline IM-250 Napadisylate has an XRPD pattern displaying at least two, at least three, at least four, at least five, at least six, at least seven, at least eight, or at least nine of the degree 2θ-reflections with the greatest intensity as the XRPD pattern substantially as shown in FIG. 8.

More preferably, such solid form IM-250 Napadisylate exhibits an X-ray powder diffraction (XRPD) pattern substantially as shown in FIG. 8.

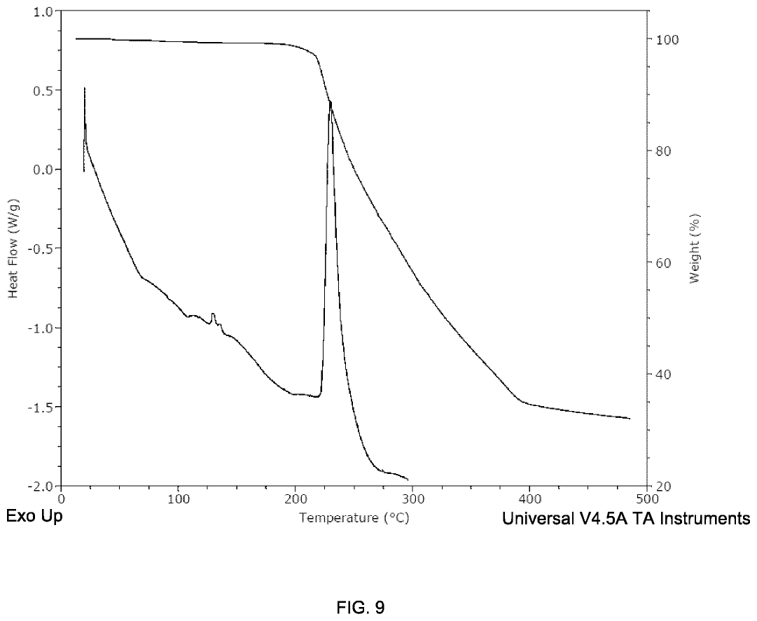

Further, such IM-250 Napadisylate may exhibit a differential scanning calorimetry (DSC) thermogram substantially as shown in FIG. 9.

Further, such IM-250 Napadisylate may exhibit a thermogravimetric analysis (TGA) thermogram substantially as shown in FIG. 9.

In some embodiments of crystalline IM-250 Napadisylate, at least one, at least two, or all of the following (a)-(c) apply: (a) crystalline IM-250 Napadisylate has an XRPD pattern substantially as shown in FIG. 8; (b) crystalline IM-250 Napadisylate has a DSC thermogram substantially as shown in FIG. 9; (c) crystalline IM-250 Napadisylate has a TGA thermogram substantially as shown in FIG. 9.

In some embodiments, crystalline IM-250 Napadisylate has at least one, at least two, or at least three of the following properties:
(a) an XRPD pattern substantially as shown in FIG. 8
(b) a DSC thermogram substantially as shown in FIG. 9
(c) a TGA thermogram substantially as shown in FIG. 9.

In some embodiments, IM-250 Napadisylate has a differential scanning calorimetry thermogram comprising an exotherm with an onset at about 223° C.

Accordingly, a solid IM-250 Napadisylate may further be characterized by having a melting point of 230° C. (±5° C.).

In some embodiments, provided herein is IM-250 Napadisylate with the structure

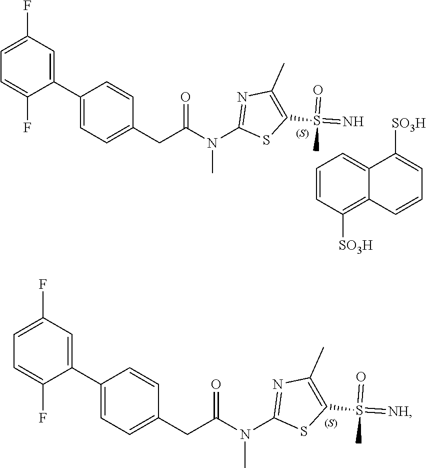

wherein naphthalene-1,5-disulfonic acid and (S)-2-(2′,5′-difluoro-[1,1′-biphenyl]-4-yl)-N-methyl-N-(4-methyl-5-(S-methylsulfonimidoyl)thiazol-2-yl)acetamide are in a 1 to 2±0.2 molar ratio.

The napadisylate salt, IM-250 Napadisylate, surprisingly turned out to exhibit several advantages with respect to chemical and physical stability, (lack of) hygroscopicity and improved bioavailability, while other tested salts, as shown in the Examples below, were less advantageous. Accordingly, a compound, IM-250 Napadisylate salt, is a particularly preferred embodiment of the present invention.

### Deuterated IM-250 Napadisylate Salt-d3-IM-250 Napadisylate

In another embodiment of the invention the IM-250 Napadisylate salt can also be present in the deuterated form of a naphthalenedisulfonic acid salt of a deuterated compound IM-250, being represented by the structure

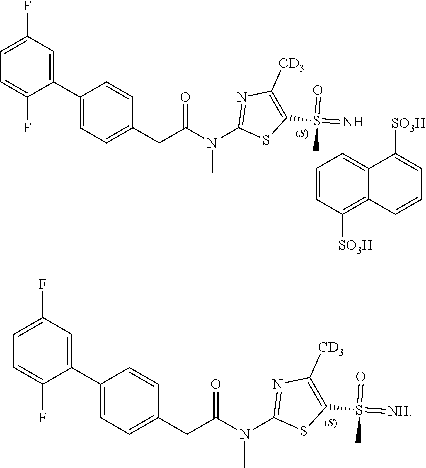

### Solid Form IM-315

A further embodiment of the invention relates to a solid (crystalline) compound according to Formula (I), which is IM-315 with the structure

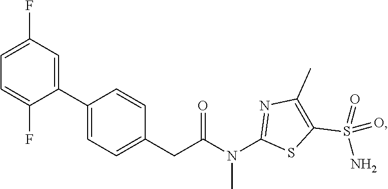 [SMILES: Cc1nc(N(C)C(=O)Cc2ccc(-c3cc(F)ccc3F)cc2)sc1S(N)(=O)=O]

wherein this solid IM-315 form is characterized by having an XRPD pattern comprising (characteristic peaks) degree 2θ-reflections (±0.2 degrees 2θ) at 6.4, 12.5 and 18.3 degrees.

In some embodiments, IM-315 is characterized by an XRPD pattern comprising degree 2θ-reflections (±0.2 degrees 2θ) at 6.4, 12.5 and 18.3 degrees and one or two of the degree 2θ-reflections (±0.2 degrees 2θ) at 22.3 and 23.1 degrees.

In some embodiments, IM-315 has an XRPD pattern comprising degree 2θ-reflections (±0.2 degrees 2θ) at 6.4, 12.5, 18.3, 22.3 and 23.1 degrees.

In some embodiments, IM-315 has an XRPD pattern comprising at least 3 of the following peaks: 6.4, 12.5, 18.3, 22.3 and 23.1 degrees 2θ (±0.2 degrees 2θ).

All values determined on a diffractometer using Cu-Kα radiation at a wavelength of 1.54 Å.

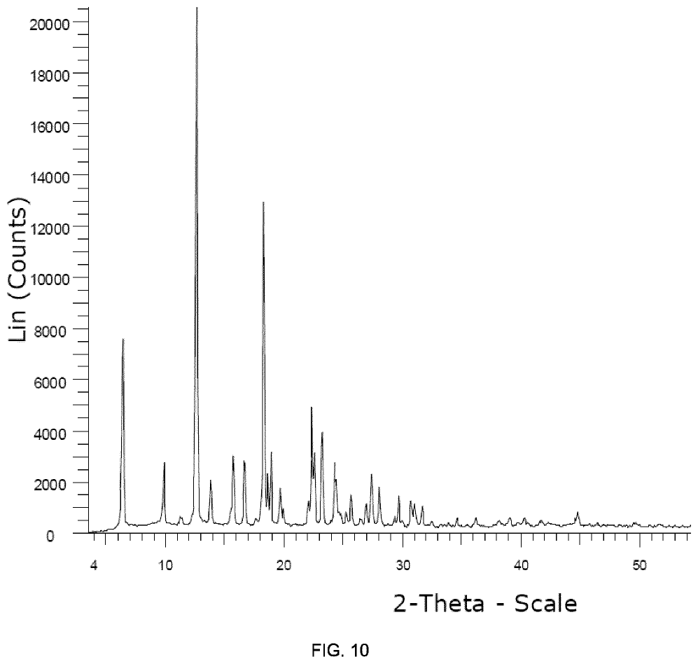

In some embodiments, crystalline IM-315 has an XRPD pattern displaying at least two, at least three, at least four, at least five, at least six, at least seven, at least eight, or at least nine of the degree 2θ-reflections with the greatest intensity as the XRPD pattern substantially as shown in FIG. 10.

More preferably, such solid form, IM-315, exhibits an X-ray powder diffraction (XRPD) pattern substantially as shown in FIG. 10.

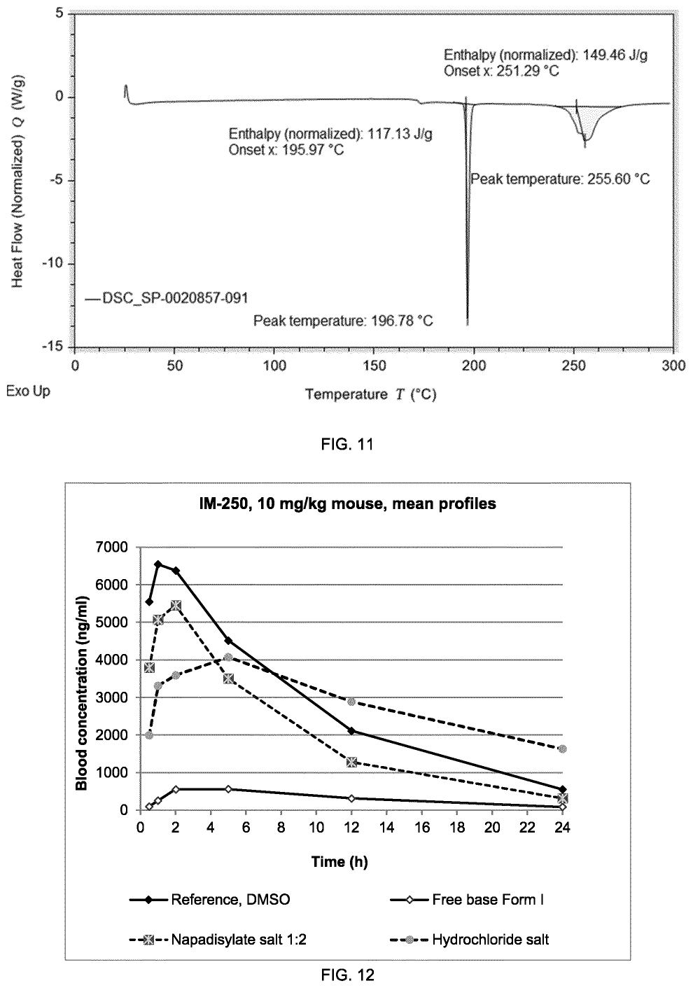

Further, such IM-315 may exhibit a differential scanning calorimetry (DSC) thermogram substantially as shown in FIG. 11.

In some embodiments of crystalline IM-315, at least one or all of the following (a)-(b) apply: (a) crystalline IM-315 has an XRPD pattern substantially as shown in FIG. 10; (b) crystalline IM-315 has a DSC thermogram substantially as shown in FIG. 11.

In some embodiments, crystalline IM-315 has at least one or at least two of the following properties:
(a) an XRPD pattern substantially as shown in FIG. 10
(b) a DSC thermogram substantially as shown in FIG. 11.

Further, such IM-315 form has a differential scanning calorimetry thermogram comprising an exotherm with an onset at about 196° C.

Accordingly, a solid IM-315 form may further be characterized by having a melting point of 197° C. (±5° C.).

As described in Example 5, the IM-315 form according to the present invention is present as a white solid.

In contrast, the Example Compound No. 87 as disclosed in WO2003/007946 and in WO2001/047904 is characterized therein by having a melting point of 184° C. and being obtained in the form of a light yellow solid. Therewith, IM-315 according to the present invention differs from the Example Compound No. 87 of WO2003/007946 and WO2001/047904 and it can be concluded that the present form IM-315 constitutes a new polymorph form, different from Example Compound No. 87 of WO2003/007946 and WO2001/047904.

### Administration Forms and Medical Use of the Solid Forms of Compounds of Formula (I)

A further aspect of the present invention relates to a pharmaceutical formulation, comprising one or more of the compounds of any of the above described embodiments.

A further aspect of the present invention relates to the compounds of any of the above described embodiments for the use as a medicament.

Particularly the invention relates to the described compounds for use in the treatment or prophylaxis of a disease or disorder associated with viral infections.

More particularly the invention relates to the described compounds for use in the treatment or prophylaxis of a disease or disorder, which is associated with viral infections caused by herpes viruses, such as in particular by Herpes simplex viruses, i.e. for the use in the treatment or prophylasis of herpes infections, such as herpex simplex infections.

In a further aspect the invention relates to the described compounds for use in treating and eliminating latent (dormant) forms of herpes viruses in neuronal tissue and nerves, preferably for avoiding or preventing recurrence and reactivation of herpes infections or even severe implications associated therewith, such as herpes simplex encephalitis (HSE).

In a further aspect the invention relates to the described compounds for use in the treatment or prophylaxis of neurodegenerative diseases caused by viruses, such as in particular Alzheimers disease caused by viruses, in particular caused by Herpes simplex viruses.

In a further aspect the invention relates to the described compounds for the use in the treatment and prophylaxis of herpes infections, in particular Herpes simplex infections, in patients displaying Herpes labialis, Herpes genitalis and Herpes-related keratitis, Alzheimers disease, encephalitis, pneumonia, hepatitis; in patients with a suppressed immune system, such as AIDS patients, cancer patients, patients having a genetic immunodeficiency, transplant patients; in new-born children and infants; in Herpes-positive patients, in particular Herpes-simplex-positive patients, in patients for suppressing recurrence (suppression therapy); or for use in patients, in particular in Herpes-positive patients, in particular Herpes-simplex-positive patients, who are resistant to nucleosidic antiviral therapy such as acyclovir, penciclovir, famciclovir, ganciclovir, valacyclovir and/or foscarnet or cidofovir.

The compounds according to the present invention are considered for the use in the prophylaxis and treatment of the respective disorders and diseases in humans as well as in animals.

Accordingly, the invention relates to the use of the compounds as described herein for the preparation of a medicament.

Further, the invention relates to a method of preventing or treating a disease or disorder associated with viral infections, such as a disease or disorder, which is associated with viral infections caused by herpes viruses, such as in particular by Herpes simplex viruses as well as a method of treating and eliminating latent (dormant) forms of herpes viruses in neuronal tissue and nerves, preferably for avoiding or preventing recurrence and reactivation of herpes infections or even severe implications associated therewith, such as herpes simplex encephalitis (HSE) or a method of preventing or treating neurodegenerative diseases caused by viruses, such as in particular Alzheimers disease, said methods comprising administering to a human or animal in need thereof an effective amount of a compound or of a composition comprising said compounds as described herein.

In practical use, the compounds used in the present invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing compositions for oral dosage forms, any of the usual pharmaceutical media may be employed, such as, for example, water, glycols, oils, alcohols, flavouring agents, preservatives, colouring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2.0 percent to about 60.0 percent of the weight of the unit. The amount of active compound in such therapeutically useful compositions is such that an effective dosage will be obtained. The active compounds can also be administered intranasally as, for example, liquid drops or spray or as eye drops.

The tablets, pills, capsules and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavouring such as cherry or orange flavour.

The compounds used in the present invention may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral (including intravenous), ocular, pulmonary, nasal and the like may be employed. Dosage forms include tablets, troches, dispersions, suspensions, solutions, capsules, creams, gels, ointments, aerosols and the like. Preferably compounds of the present invention are administered orally or topical as eye drops, creams or gels, more preferably the compounds of the present invention are administered orally.

The effective dosage of active ingredient employed may vary depending on the particular compound employed, the mode of administration, the condition being treated and the severity of the condition being treated. Such dosage may be ascertained readily by a person skilled in the art.

The compounds of the present invention may also be present in combination with further active ingredients, in particular with one or more active ingredients exhibiting advantageous effects in the treatment of any of the disorders or diseases as described herein. Very particularly the compounds of the present invention are present in a composition in combination with at least one further active substance being effective in treating a disease or disorder associated with viral infections (antiviral active compounds), preferably a disease or disorder being associated with viral infections caused by herpes viruses, such as in particular by Herpes simplex viruses, thus relating to a so called combination therapy. The at least one further active substance (immune modulators, e.g. glucocorticoids) being effective in treating a disease or disorder associated with viral infections or more preferably antiviral active compounds selected from the group consisting of nucleosidic drugs such as acyclovir, valacyclovir, penciclovir, ganciclovir, famciclovir and trifluridine, as well as compounds such as foscarnet and cidofovir.

Accordingly, the present invention further relates to a pharmaceutical composition comprising one or more of the compounds as described herein and at least one pharmaceutically acceptable carrier and/or excipient and/or at least one further active substance being effective in treating a disease or disorder associated with viral infections (antiviral active compounds).

A further aspect of the invention relates to the use of the compounds described herein, which act as helicase primase inhibitors, in a combination therapy with oncolytic viruses for treating tumors, cancer or neoplasia.

A further embodiment of this additional aspect of the invention relates to a pharmaceutical composition for the use as an antidote in a combination therapy with oncolytic viruses for treating cancer, which comprises at least one helicase primase inhibitor as defined in any embodiment described herein, which acts to control, modulate, inhibit or shut off the activity of oncolytic viruses sensitive to said inhibitors used in cancer therapy, and which may further comprise at least one pharmaceutically acceptable carrier and/or excipient and/or at least one further active substance, such as antiviral active or immune modulating compounds, including checkpoint inhibitors, being effective in treating a disease or disorder associated with oncolytic viral infections used in the treatment of cancer.

A further embodiment of this additional aspect of the invention relates to the helicase primase inhibitor compounds or the pharmaceutical compositions of the present invention for the use in a combination therapy with oncolytic viruses as described in detail in WO2020/109389, wherein the cancer to be treated is solid cancer, preferably the cancer disease is selected from liver cancer, lung cancer, colon cancer, pancreas cancer, kidney cancer, brain cancer, melanoma and glioblastoma etc.

A further embodiment of this additional aspect of the invention relates to the helicase primase inhibitor compounds or the pharmaceutical compositions of the present invention for the use in a combination therapy with oncolytic viruses as described in WO2020/109389, wherein the oncolytic viruses are oncolytic herpesviruses.

A further embodiment of this additional aspect of the invention relates to the helicase primase inhibitor compounds or the pharmaceutical compositions of the present invention for the use in a combination therapy with oncolytic viruses as described in WO2020/109389, wherein the cancer therapy comprises infusion, injection, intratumoral injection or topical or transdermal application of the oncolytic viruses or oncolytic virus infected cells and/or of the helicase primase inhibitors or the pharmaceutical composition comprising the same.

A further embodiment of this additional aspect of the invention relates to the helicase primase inhibitor compounds or the pharmaceutical compositions of the present invention for the use in a combination therapy with oncolytic viruses as described in WO2020/109389, wherein the oncolytic viruses or oncolytic viruses infected cells are selected from an oncolytic wildtype, a clinical isolate or a laboratory herpesvirus strain or a genetically engineered or multi-mutated optionally attenuated or boosted oncolytic herpesvirus.

A further embodiment of this additional aspect of the invention relates to a kit comprising at least one of the helicase primase inhibitor compounds or the pharmaceutical composition of the present invention for the use in a combination therapy with oncolytic viruses as described in WO2020/109389, and at least one oncolytic virus selected from a wildtype, a laboratory strain, a clinical isolate and a genetically engineered or multi-mutated oncolytic virus.

A further embodiment of this additional aspect of the invention relates to said kit for the use in the treatment of cancer as defined herein.

The helicase primase inhibitor compounds, pharmaceutical compositions or kits for the use in a combination therapy with oncolytic viruses as described herein may be applied to one or more of the following patient groups: infants; herpes-positive patients, in particular oncolytic herpes-simplex-positive patients, for suppressing recurrence or oncolytic viral shedding; patients, in particular herpes-positive patients, in particular oncolytic herpes-simplex-positive patients, who are resistant to nucleosidic antiviral therapy such as acyclovir, penciclovir, famciclovir, ganciclovir, valacyclovir and/or foscarnet or cidofovir.

A further aspect of the present invention relates to the preparation of compounds having the following structure:

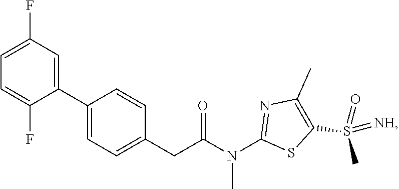 [SMILES: Cc1nc(N(C)C(=O)Cc2ccc(-c3cc(F)ccc3F)cc2)sc1[S@@](C)(=N)=O]

or a pharmaceutically acceptable salt, co-crystal, hydrate or solvate thereof,
(a) comprising a step of contacting a compound P2b:

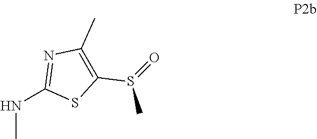

with a compound:

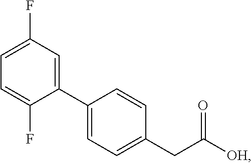 [SMILES: O=C(O)Cc1ccc(-c2cc(F)ccc2F)cc1]

(b) a step of contacting a compound P2c:

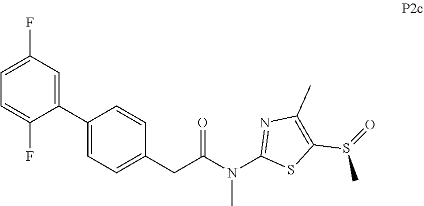 [SMILES: Cc1nc(N(C)C(=O)Cc2ccc(-c3cc(F)ccc3F)cc2)sc1[S@](C)=O]

with Rh₂(OAc)₄, tert-butyl carbamate, magnesium oxide and (diacetoxy) iodobenzene, under conditions sufficient to form a compound P2d:

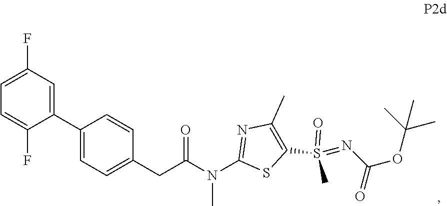 [SMILES: Cc1nc(N(C)C(=O)Cc2ccc(-c3cc(F)ccc3F)cc2)sc1[S@](C)(=O)=NC(=O)OC(C)(C)C]

(c) a step of deprotecting the compound P2d under conditions sufficient to form a compound having the structure

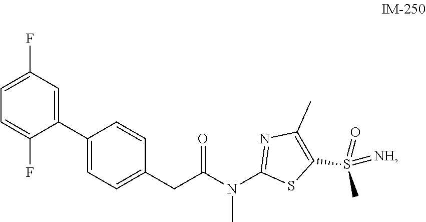 [SMILES: Cc1nc(N(C)C(=O)Cc2ccc(-c3cc(F)ccc3F)cc2)sc1[S@@](C)(=N)=O]

and
(d) optionally converting the compound IM-250 into a pharmaceutically acceptable salt, co-crystal, hydrate or solvate thereof.

A further embodiment relates to the process described above, wherein compound P2d:

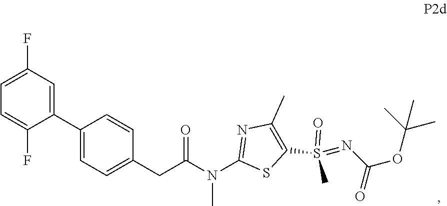 [SMILES: Cc1nc(N(C)C(=O)Cc2ccc(-c3cc(F)ccc3F)cc2)sc1[S@](C)(=O)=NC(=O)OC(C)(C)C]

is deprotected in step (c) with hydrochloric acid to form a HCl salt of a the compound IM-250, corresponding to the compound IM-250 HCl salt:

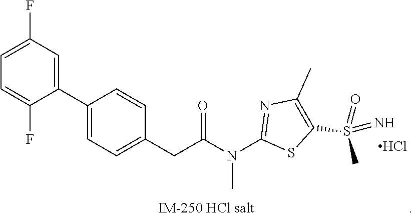 [SMILES: Cc1nc(N(C)C(=O)Cc2ccc(-c3cc(F)ccc3F)cc2)sc1[S@@](C)(=N)=O ; Cl]

In a preferred embodiment the IM-250 HCl salt is recrystallized from isopropanol or ethanol, preferably from ethanol.

In another embodiment in the process described above, the compound P2d:

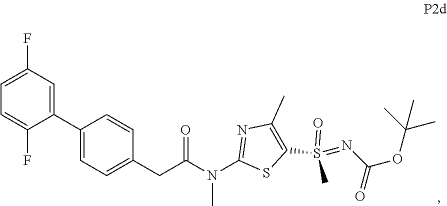 [SMILES: Cc1nc(N(C)C(=O)Cc2ccc(-c3cc(F)ccc3F)cc2)sc1[S@](C)(=O)=NC(=O)OC(C)(C)C]

is deprotected in step (c) with 1,5-naphthalenedisulfonic acid tetrahydrate to form a napadisylate salt of the compound IM-250.

In a similar way the corresponding deuterated compounds can be obtained, including a step of deuteration at the position corresponding to Y in the Formula (I).

### EXPERIMENTAL PART

### Abbreviations

HPMC hydroxypropylmethylcellulose
DMF dimethylformamide
DMSO dimethylsulfoxide
DSC differential scanning calorimeter
EA ethyl acetatee
EDCl·HCl 1-(3-dimethylaminopropyl)-3-ethylcarbodiimide hydrochloride
FCC flash column chromatography on silica gel
HOBt 1-hydroxybenzotriazol
PE petroleum etherl
rt room temperature
SFC supercritical fluid chromatography
TFA trifluoroacetic acid
TGA thermogravimetric analysis
THF tetrahydrofuran
XRPD X-ray powder diffraction

### Experimental Section

### X-Ray Powder Diffraction (XRPD)

XRPD analysis was performed on a Bruker D2 Phase diffractometer using a copper anti-cathode, a mono-crystalline silicone sample holder and a position sensitive detector (LynxExe). Powder sample was loaded on a flat mono-crystalline silicone sample holder in a way to avoid preferred orientation and to sensure planarity of the speciem surface. Instrument operation conditions were as follows: ambient temperature and atmosphere; X-ray generator voltage 30 kV and intensity 10 mA; X-ray source: target Copper; emission radiation Kα₁=0.15406 nm, Kα₂=0.15444 nm, ratio Kα₂/Kα₁=0.5, KB filter radiation Nickel; Slit: anti-divergence 1 nM, Sollers slit 2.5°; Goniometer: angular sector analyzed from 4° to 45° or 4° to 50° for 20, step size 0.07° for 20; Sample holder rotation speed: 30 rpm; Detection: exposition time per step size of goniometer 1 s.

### Differential Scanning Calorimeter (DSC)

DSC analysis was performed on a Q1000 TA Instruments analyzer. The sample to be analyzed was weighed in an aluminium capsule, which was then crimped and put into the calorimeter oven. Instrument operation conditions were as follows: heater ramp 10° C./min; final temperature 230° C. or 240° C.; carrier gas: nitrogen (Messer “qualité Azote 5.0”) with flow rate of 50 mL/min.

### Thermogravimetric Analysis (TGA)

TGA analysis was performed on a TA Instruments TGA Hi-Res 2950. The sample was placed in an opened aluminium basket and analysed as follows: mass assay 5 mg; heating ramp 10° C./min; final temperature 500° C.; carrier gas: nitrogen (Messer “qualité Azote 5.0”) with flow rate of 95-105 mL/min.

### Example 1: Synthesis of IM-250 Free Base Form I

The title compound was prepared by separation of the racemic mixture (as described in Example 7 of WO2019/068817) by chiral SFC chromatography, using as stationary phase Chiralcel OJ and as mobile phase 60/40 vol. % CO₂/IPA and additional following data:
Instrument: SFC-200 (Thar, Waters)
Column: OJ 20×250 mm, 10 μm (Daicel)
Column temperature: 35° C.
Flow rate: 100 g/min
Back pressure: 100 bar
Detection wavelength: 214 nm
Cycle time: 6 min
Sample solution: 70 g dissolved in 2000 mL dichloromethane
Injection volume: 3 mL

The title compound IM-250 Free base Form I is obtainable by removing the mobile phase (solvent) of the first eluting enantiomer (retention time: 3.25 min) after evaporation of the CO₂ and removing the IPA by a rotary evaporator at 40° C.

XRPD analysis was conducted. FIG. 1 shows a XRPD pattern of IM-250 Free base Form I. XRPD peaks were identified and are included in Table 1 below.

TABLE 1
XRPD Peak positions (°2Θ) and intensities

| °2-Theta (°2Θ) | Relative intensity (%) |
|---|---|
| 7.7 | 11 |
| 9.1 | 46 |
| 9.2 | 64 |
| 13.3 | 25 |
| 13.7 | 100 |
| 13.8 | 76 |
| 14.4 | 54 |
| 15.3 | 15 |
| 15.5 | 19 |
| 16.8 | 10 |
| 18.1 | 10 |
| 18.7 | 72 |
| 19.3 | 22 |
| 23.4 | 15 |
| 24.0 | 39 |
| 24.6 | 18 |
| 26.0 | 22 |
| 26.4 | 29 |
| 26.7 | 28 |
| 27.3 | 37 |
| 30.6 | 10 |

TGA and DSC analysis were performed. FIG. 2 shows an overlay of the DSC and TGA thermogram of IM-250 Free base Form I. The TGA analysis (right curve) showed that solids lost about 0.1% weight below about 160° C. and that solids lost about 76% weight from about 240-300° C. (decomposition). The DSC analysis revealed an endotherm with onset at about 163° C. and peak at 164° C. (transition enthalphy-83 J/g).

### Example 2: Synthesis of IM-250 Free Base Form III

### Step 1: N,4-Dimethyl-5-(methylsulfinyl)thiazol-2-amine (P2a)

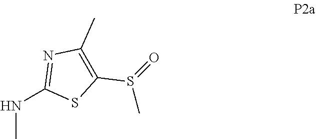

To a solution of N,4-dimethyl-5-(methylthio)thiazol-2-amine (as described in Example P4a of WO2019/068817) (80 g, 0.46 mol) in CH₂Cl₂ (1.0 L) meta-chloroperoxybenzoic acid (83 g, 85%, 0.46 mol) was added at rt and the mixture was stirred for 30 min. Then a saturated NaHCO₃ solution was added. The mixture was extracted with CH₂Cl₂ (1.5 L) and washed with brine (500 mL). The organic layer was dried over Na₂SO₄, filtered, concentrated and purified by FCC (CH₂Cl₂:MeOH=10:1) to give the title compound P2a (58 g, 66%) as a yellow solid.

### Step 2: (−)-(S)—N,4-Dimethyl-5-(methylsulfinyl)thiazol-2-amine (P2b)

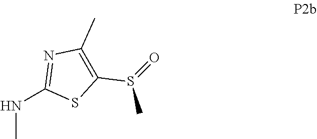

The title compound P2b was prepared by separation of the racemic mixture P2a by chiral SFC chromatography, using the following equipment and conditions:
Instrument: SFC-80 (Thar, Waters)
Column: IC 20×250 mm, 10 μm (Daicel)
Column temperature: 35° C.
Mobile phase: CO₂/MeOH with 0.2% NH₃=65/35
Flow rate: 80 g/min
Back pressure: 100 bar
Detection wavelength: 214 nm
Cycle time: 5.6 min
Sample solution: 58 g dissolved in 1 L
Injection volume: 1.5 mL

Compound P2b was obtained as a pale yellow solid (22.5 g) as the first eluting enantiomer (retention time: 2.38 min).

It has a negative optical rotation of [α]²⁰_{589 nm}−33.5° (c=1.00 g/100 mL MeOH).

¹H-NMR (DMSO-d₆, 400 MHZ): 8.22 (d, J=4.4 Hz, 1H), 2.84 (d, J=4.8 Hz, 3H), 2.79 (s, 3H), 2.24 (s, 3H). MS found: 191.2 [M+H]⁺.

### Step 3: (S)-2-(2′,5′-Difluoro-[1,1′-biphenyl]-4-yl)-N-methyl-N-(4-methyl-5-(methylsulfinyl)thiazol-2-yl)acetamide (P2c)

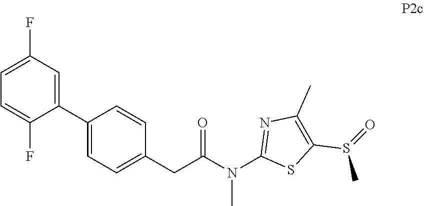 [SMILES: Cc1nc(N(C)C(=O)Cc2ccc(-c3cc(F)ccc3F)cc2)sc1[S@](C)=O]

2-(2′,5′-Difluoro-[1,1′-biphenyl]-4-yl)acetic acid (96.4 g, 389 mmol) and HOBt (78.7 g, 583 mmol) were dissolved in DMF (800 mL). The mixture was stirred at rt for 30 min, then compound P2b (74.0 g, 389 mmol) and EDCl·HCl (112 g, 583 mmol) were added. The mixture was stirred overnight, concentrated in vacuo, redisolved in EA (1.0 L) and washed with water (2×0.5 L). The organic layer was dried over Na₂SO₄, concentrated in vacuo and purified by FCC (PE:EA=1:2) to give compound P2c (145 g, 89%) as a white solid.

¹H-NMR (DMSO-d₆, 400 MHZ): 7.57 (dd, J=8.0, 1.6 Hz, 2H), 7.46-7.36 (m, 4H), 7.30-7.24 (m, 1H), 4.24 (s, 2H), 3.74 (s, 3H), 2.91 (s, 3H), 2.41 (s, 3H). MS found: 421.1 [M+H]⁺.

### Step 4: (S)-2-(2′,5′-Difluoro-[1,1′-biphenyl]-4-yl)-N-methyl-N-(4-methyl-5-(S-methyl-N-((1,1-dimethylethoxy)carbonyl)sulfonimidoyl)thiazol-2-yl)acetamide (P2d)

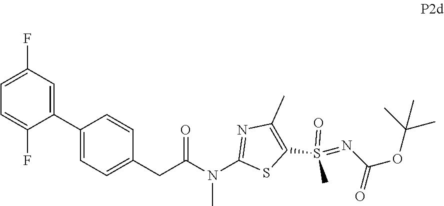 [SMILES: Cc1nc(N(C)C(=O)Cc2ccc(-c3cc(F)ccc3F)cc2)sc1[S@](C)(=O)=NC(=O)OC(C)(C)C]

Magnesium oxide (55.2 g, 1.38 mol), tert-butyl carbamate (80.7 g, 690 mmol), Rh₂ (OAc) 4 (14.5 g, 32.8 mmol) and (diacetoxy) iodobenzene (167 g, 517 mmol) were added to a solution of compound P2c (145 g, 345 mmol) in CH₂Cl₂ (1.0 L). The mixture was stirred at 40° C. for 1 hour. Additional Rh₂ (OAc) 4 (4.8 g, 11 mmol), MgO (18.4 g, 460 mmol), tert-butyl carbamate (26.9 g, 230 mmol) and (diacetoxy) iodobenzene (55.5 g, 172 mmol) were added and stirred overnight. Then the mixture was filtered through a pad of celite, the solvent was removed under reduced pressure and the crude product was purified by FCC (PE:EA=1:1) to give compound P2d (160 g, 87%) as white solid.

¹H-NMR (DMSO-d₆, 400 MHZ): 7.57 (dd, J=8.0, 1.2 Hz, 2H), 7.45-7.35 (m, 4H), 7.29-7.24 (m, 1H), 4.26 (s, 2H), 3.75 (s, 3H), 3.47 (s, 3H), 2.51 (s, 3H), 1.32 (s, 9H). MS found: 536.1 [M+H]⁺.

### Step 5: (S)-2-(2′,5′-Difluoro-[1,1′-biphenyl]-4-yl)-N-methyl-N-(4-methyl-5-(S-methylsulfon-imidoyl)thiazol-2-yl)acetamide (IM-250 Free Base)

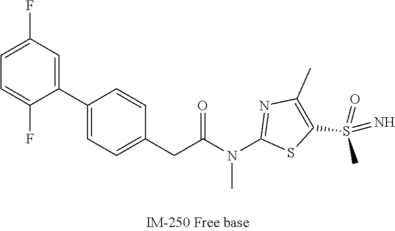 [SMILES: Cc1nc(N(C)C(=O)Cc2ccc(-c3cc(F)ccc3F)cc2)sc1[S@@](C)(=N)=O]

At ambient temperature, compound P2d (160 g, 299 mmol) was added to a stirred solution of TFA (80 mL) acid in CH₂Cl₂ (0.5 L) and stirring was continued for 90 min. The mixture was concentrated, then resolved in CH₂Cl₂, washed with saturated NaHCO₃ solution (3×0.5 L), dried over Na₂SO₄, concentrated and purified by FCC (PE:EA=1:2) to give IM-250 Free base Form III (120 g, 92%) as a white solid after removing the organic solvents on a rotary evaporator at 30° C.

XRPD analysis was conducted. FIG. 3 shows a XRPD pattern of IM-250 Free base Form III. XRPD peaks were identified and are included in Table 2 below.

TABLE 2
XRPD Peak positions (°2Θ) and intensities

| °2-Theta (°2Θ) | Relative intensity (%) |
|---|---|
| 9.7 | 28 |
| 12.3 | 79 |
| 12.9 | 40 |
| 13.6 | 12 |
| 15.6 | 100 |
| 16.1 | 21 |
| 17.4 | 28 |
| 18.3 | 12 |
| 19.4 | 18 |
| 20.3 | 11 |
| 20.9 | 16 |
| 21.4 | 19 |
| 21.7 | 12 |
| 22.7 | 34 |
| 23.0 | 10 |
| 23.8 | 32 |
| 24.7 | 10 |
| 26.0 | 23 |
| 27.4 | 17 |
| 27.9 | 17 |
| 29.0 | 14 |
| 29.5 | 11 |

TGA and DSC analysis were performed. FIG. 4 shows an overlay of the DSC and TGA thermogram of IM-250 Free base Form III. The TGA analysis (right curve) showed that solids lost about 0.2% weight below about 130° C. and that solids lost about 75% weight from about 240-300° C. (decomposition). The DSC analysis revealed an endotherm with onset at about 141° C. and peak at 143° C. (transition enthalphy-83 J/g), an exotherm with peak at 148° C. (transition enthalphy 43 J/g) for recrystallization and an endotherm with onset at about 163° C. and peak at 164° C. (transition enthalphy-52 J/g).

### Example 3: Synthesis of IM-250 HCl Salt

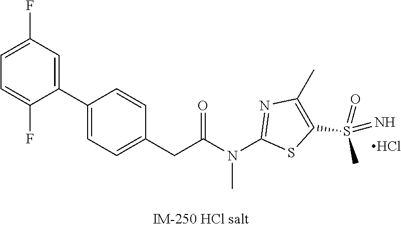 [SMILES: Cc1nc(N(C)C(=O)Cc2ccc(-c3cc(F)ccc3F)cc2)sc1[S@@](C)(=N)=O ; Cl]

IM-250 Form I (205 mg, 470 μmol) was solubilized in acetone (5 mL) by stirring on a rotary evaporator at 50° C. and atmospheric pressure. A volume of 1N HCl corresponding to a 1:1 stoichiometry was the added. The solvent was then evaporated to dryness at 50° C. leading to a film. This film was resuspended and solubilized in EtOH (4 mL) at rt. The solvent was then evaporated to dryness at 50° C. leading to a meringue. This film was resuspended and solubilized in isopropanol (1 mL) at 50° C., kept at rt leading to a partial demixing (after approx. 30 min) and then warmed again to 50° C. for resolubilization. Very quickly, a strong crystallization occurred. One additional heating (50° C.) and cooling (rt) cycle was performed (each 20 min) and the the sample was kept at rt for 2 days. The supernatant solvent was removed and the powder was finally dried under a dynamic vacuum (70° C. for 40 min) to obtain IM-250 HCl salt as colorless crystals.

XRPD analysis was conducted. FIG. 5 shows a XRPD pattern of IM-250 HCl salt. XRPD peaks were identified and are included in Table 3 below.

TABLE 3
XRPD Peak positions (°2Θ) and intensities

| °2-Theta (°2Θ) | Relative intensity (%) |
|---|---|
| 9.1 | 24 |
| 11.2 | 13 |
| 11.8 | 20 |
| 13.7 | 100 |
| 14.1 | 17 |
| 16.4 | 23 |
| 17.0 | 55 |
| 17.3 | 21 |
| 17.7 | 98 |
| 18.2 | 34 |
| 19.4 | 26 |
| 19.8 | 42 |
| 21.1 | 23 |
| 21.3 | 33 |
| 21.8 | 33 |
| 22.6 | 15 |
| 22.8 | 62 |
| 23.4 | 11 |
| 23.7 | 11 |
| 24.0 | 15 |
| 24.2 | 16 |
| 25.0 | 30 |
| 26.3 | 35 |
| 26.9 | 12 |
| 27.1 | 19 |
| 27.4 | 11 |
| 27.7 | 22 |
| 32.0 | 11 |

TGA and DSC analysis were performed. FIG. 6 shows an overlay of the DSC and TGA thermogram of IM-250 HCl salt. The TGA analysis (right curve) showed a mass loss of 9.8% upon heating with an onset/endset temperature of 151/170° C. before the main thermal decomposition can be detected with an onset temperature of 221° C. The 9.8% mass loss can be attributed to the departure of the HCl moiety. The DSC analysis revealed no true melting point. The not resolved double endothermic events observed from 160° C. are concomitant to the loss of the HCl moiety observed on the TGA profile.

Alternative Synthesis of IM-250 HCl Salt with EtOH:

IM-250 Form III (4.75 g) was solubilized in acetone (150 mL) by stirring on a rotary evaporator at rt and atmospheric pressure. A volume of 1N HCl corresponding to a 1:1 stoichiometry was then added. The solvent was partially evaporated (about 100 mL) at 50° C. To better trap the water brought by the HCl solution, EtOH (50 mL) was added to the solution before performing a new evaporation up to a remaining volume of a few milliliters (syrupy liquid). The sample was then brought back at rt, leading to a beginning of crystallization. Additional EtOH (50 mL) was then added to the sample (always in order to help better removing the water brought with the HCl solution) leading to a non-expected increase of the crystallization. XRPD analysis was conducted. FIG. 7 shows a XRPD pattern of IM-250 HCl salt. The XRPD peaks identified were similar as shown in FIG. 5, indicating that the same HCl polymorph was produced.

### Alternative Synthesis of IM-250 HCl Salt Via Direct Boc-Deprotection:

Compound P2d was dissolved in acetone (10 eq.), heated to 50° C. and then HCl (4N in dioxane, 4 eq.) was added. After complete conversion, the mixture was allowed to reach rt, filtered and washed with acetone. The product was dried in vacuo at 50° C. and slurried in 3.5% aqueous HCl solution (10 eq.) at rt for 1 h, then filtered, washed with a 3.5% aq. HCl solution (10 eq.) and dried in vacuo at 50° C. to obtain the crude HCl salt, which was recrystallized in EtOH to obtain pure IM-250 HCl salt.

### Example 4: Synthesis of IM-250 Napadisylate

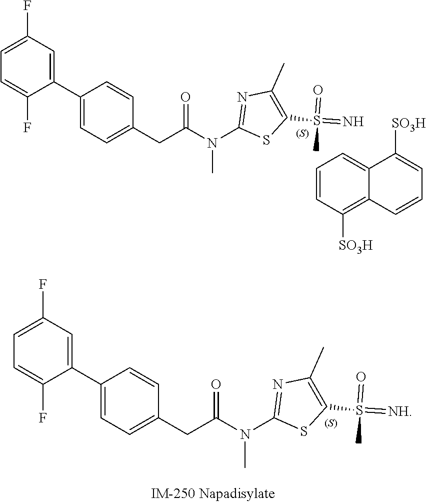

IM-250 Form I (150 mg, 344 μmol) and 1,5-naphthalenedisulfonic acid tetrahydrate (124 mg, 344 μmol) are weighted in a glass vial. A mixture of MeOH (5 mL) and THF (5 mL) was added and the suspension was stirred on a rotary evaporator at 50° C. under atmospheric pressure until complete solubilisation. The solvents are then evaporated to dryness at 50° C. to obtain a film. This film is resuspended in a mixture of water (1 mL) and EtOH (1 mL) and stirred at 40° C. under atmospheric pressure for a few minutes leading to a partial solubilization. An additional mixture of water (1 mL) and EtOH (1 mL) was added leading to a complete solubilization after about 20 min of stirring at 55° C. under atmospheric pressure. Very quickly, a strong crystallization of small particles occur. Two cycles of heating (15 min at 60° C.) and cooling (15 min at rt) are then performed. Finally the sample was kept at rt overnight. The supernatant was removed from the flask. The powder was dried at rt for 15 min and finally under vacuum for 45 min at 70° C. to obtain IM-250 Napadisylate as colorless crystals.

XRPD analysis was conducted. FIG. 8 shows a XRPD pattern of IM-250 Napadisylate. XRPD peaks were identified and are included in Table 4 below.

TABLE 4
XRPD Peak positions (°2Θ) and intensities

| °2-Theta (°2Θ) | Relative intensity (%) |
|---|---|
| 9.1 | 38 |
| 11.0 | 10 |
| 13.4 | 33 |
| 14.5 | 100 |
| 14.9 | 19 |
| 15.6 | 39 |
| 16.5 | 30 |
| 17.0 | 20 |
| 17.3 | 15 |
| 17.6 | 13 |
| 18.1 | 58 |
| 18.5 | 10 |
| 19.1 | 37 |
| 20.3 | 14 |
| 20.7 | 28 |
| 20.9 | 37 |
| 21.3 | 15 |
| 21.8 | 17 |
| 22.3 | 36 |
| 22.7 | 11 |
| 23.2 | 28 |
| 23.6 | 16 |
| 24.5 | 28 |
| 24.8 | 13 |
| 25.3 | 10 |
| 25.4 | 12 |
| 26.2 | 11 |
| 26.7 | 20 |
| 27.3 | 16 |

TGA and DSC analysis were performed. FIG. 9 shows an overlay of the DSC and TGA thermogram of IM-250 Napadisylate. The TGA analysis (right curve) showed that solids lost about 0.8% weight below about 110° C. and that solids lost about 67% weight from about 150-370° C. (decomposition). The DSC analysis revealed an exotherm with onset at about 223° C. and peak at 230° C. (transition enthalphy 152 J/g).

### Comparative Example 4: Synthesis of Additional IM-250 Salt Forms

Several additional strong acids (e.g. hydrobromic acid, sulfuric acid, camphorsulfonic acid, 1,2-ethanesulfonic acid, toluenesulfonic acid, nitric acid, methanesulfonic acid, 2-naphthalenesulfonic acid) were tested in micro-crystallization experiments in different crystallization media like water or pure organic solvents (e.g. methanol, acetonitrile, isopropyl alcohol, ethanol, acetone, tetrahydrofuran) or mixtures of these organic solvents with water (50/50 v/v). The counter-ions were tested at the 1/1 (IM-250 Free base Form I/counter-ion) molar ratio. After the crystallization experiments, the samples (corresponding to all counter-ion/crystallization medium couples) were analyzed to identify the “counter-ion/crystallization medium” couples that have led to crystallization. This allowed defining “crystallization hits”, which were then further characterized.

Macroscopic observation of the screen plates evidenced that the tested acids when combined to IM-250 Free base Form I lead to only a few solid residues. Moreover, no clearly visible crystal morphology were observed in any of these samples. By contrast, solid residues with a significant quantity of material were observed for IM-250 Free base Form I samples recrystallized alone in the different media (solvents or solvent/water mixtures). E.g. for hydrobromic acid and nitric acid, the combination with IM-250 Free base Form I lead to solid residues with a significant quantity of material in only a few samples. For sulfuric acid, ethanedisulfonic acid, toluenesulfonic acid and methanesulfonic acid, the combination with IM-250 Free base Form I lead to liquid/vitreous residues with solid residue only in a few samples. For camphorsulfonic acid and naphthalenesulfonic acid, the combination with IM-250 Free base Form I lead to liquid/vitreous residues with only a few solid residues or without solid residue. For each of these tested counter-ions, the number of crystallization occurrences and the quality of the resulting materials were then taken into account to select, for each counter-ion, a relevant sample to be further characterized (also taking into account the proper reference samples) and finally upscaled. A certain number of “IM-250 Free base Form I/counter-ion/crystallization medium” couples were selected for being observed by optical microscopy under cross-polarized light. The morphology of the crystals (when well defined crystal shapes are observed) were compared between different samples and analyzed by XRPD. From the many possibilities, only a few promising XRPDs were obtained and upscaled for further investigation of developability. In the following paragraphs, some representative attempts are outlined:

### IM-250 Hydrobromide Salt

About 150 mg of IM-250 Free base Form I were first solubilized in 5 mL of acetone (stirring on a rotary evaporator at 30° C. and atmospheric pressure). A volume of 48% hydrobromic acid corresponding to a 1:1 stoichiometry was then added. Solvent was then evaporated to dryness at 40° C. leading to a crust/meringue. The film was re-suspended and solubilized in 6 mL of THF at room temperature and atmospheric pressure, leading very quickly to a strong crystallization.

Several cycles (2) of heating (15 min at 40° C.) and cooling (15 min at room temperature) were then performed to tentatively increase the size and quality of crystals.

The sample was then kept at room temperature for a few hours. As only a small volume of supernatant could be easily removed (the crystals occupying the whole initial solution mixture), the sample was filtered under vacuum. The isolated powder was finally dried under dynamic vacuum for 10 minutes at room temperature and then about 30 minutes at 60° C. Microscopy pictures of the isolated sample displayed high birefringence of particles, when observed between crossed polarizer and analyzer, indicated the sample was well crystallized.

The overlaid XRPD profiles evidenced that the sample of IM-250 Free base crystallized with 1 eq hydrobromide acid exhibit a XPRD pattern, which differs from the one of the parent form of IM-250 Free base Form I.

Upon heating, different mass losses were detected in the TGA profile before the main thermal decomposition detected with an onset temperature of 223° C.: 1) A first mass loss of 0.5%, detected with onset/endset temperatures of 17/25° C., could correspond to the departure of water and/or solvent that would simply be adsorbed on the powder; 2) A second mass loss of 3.5% detected with onset/endset temperatures of 78/85° C.; 3) A third mass loss of 5.7% detected with onset/endset temperatures of 103/115° C.; and 4) A fourth mass loss of 5.7% detected with onset/endset temperatures of 124/130° C.

The FTIR analysis of evolved gas from TGA analysis showed that THF FTIR spectrum best matches with the FTIR spectrum of the volatile species leaving the sample during the mass losses of 3.5% and 5.7%. The hypothesis of a solvated form crystallization was therefore likely.

The percentage of IM-250 Free base in the salt sample, determined by comparison with the freebase was found to be 78.1% by HPLC (to be compared to the 84.3% of the theoretical IM-250 Free base percentage in the targeted IM-250 hydrobromide salt of 1:1 stoichiometry).

This result, lower than what would be expected, confirms this sample is likely crystallized into a solvated form.

The HPLC profile shows a very slight degradation of the active ingredient in the isolated solid with the appearance of a few new impurities (% purity at 285 nm=99.5%, to be compared to the % purity=99.8% of the parent freebase).

In conclusion, crystalline samples of IM-250 hydrobromide salt have been obtained in THF. Unfortunately, results from HPLC and TGA-FTIR analyses evidence the produced hydrobromide salt is actually a THF-solvate, which makes it non-suitable for development. The experiments were repeated with a second batch and delivered the same outcome.

This comparative experiments for the IM-250 hydrobromide salt highlight, that from the many possibilities to generate crystals within the “IM-250 Free base/counter-ion/crystallization medium”-matrix surprisingly only few setups finally furnished IM-250 salt forms suitable for further development as medicament, such as for example IM-250 Napadisylate and IM-250 HCl salt.

### Example 5: Synthesis of crystalline 2-(2′,5′-Difluoro-[1,1′-biphenyl]-4-yl)-N-methyl-N-(4-methyl-5-sulfamoylthiazol-2-yl)acetamide IM-315

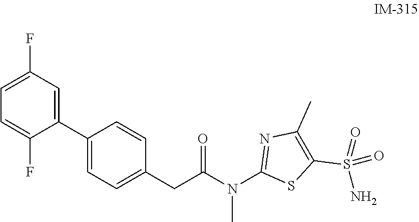 [SMILES: Cc1nc(N(C)C(=O)Cc2ccc(-c3cc(F)ccc3F)cc2)sc1S(N)(=O)=O]

2-(2′,5′-Difluoro-[1,1′-biphenyl]-4-yl)acetic acid (22.0 g, 88.7 mmol) and HOBt (18.0 g, 133 mmol) was dissolved in DMF (0.4 L) and the mixture was stirred at rt for 30 min. Then 4-methyl-2-(methylamino) thiazole-5-sulfonamide (18.4 g, 88.7 mmol) and EDCl·HCl (26.0 g, 133 mmol) was added. The mixture was stirred at rt overnight, diluted with EtOAc (0.5 L) and washed with water (2×250 mL) and brine. The organic layer was dried over Na₂SO₄, concentrated and purified by FCC (PE:EA=1:2) to give IM-315 (25.2 g, 65%) as a white solid.

¹H NMR (400 MHZ, DMSO-d₆) δ: 7.65 (s, 2H), 7.60-7.52 (m, 2H), 7.46-7.33 (m, 4H), 7.30-7.22 (m, 1H), 4.23 (s, 2H), 3.72 (s, 3H), 2.48 (s, 3H). MS found: 438.0 [M+H]⁺.

XRPD analysis was conducted. FIG. 10 shows a XRPD pattern of IM-315. XRPD peaks were identified and are included in Table 5 below.

TABLE 5
XRPD Peak positions (°2Θ) and intensities

| °2-Theta (°2Θ) | Relative intensity (%) |
|---|---|
| 6.4 | 37 |
| 9.8 | 13 |
| 12.5 | 100 |
| 13.8 | 10 |
| 15.7 | 15 |
| 16.6 | 14 |
| 18.3 | 63 |
| 18.6 | 13 |
| 18.8 | 15 |
| 22.3 | 24 |
| 22.6 | 15 |
| 23.1 | 19 |
| 24.3 | 13 |
| 24.4 | 10 |
| 27.4 | 11 |

DSC analysis were performed. FIG. 11 shows the DSC thermogram of IM-315. The DSC analysis revealed an exotherm with onset at about 196° C. and peak at 197° C. (transition enthalphy 117 J/g). Noteworthy, this melting point is higher compared to the stated melting point of 184° C. described for Example 8 of WO2003/007946 and Exp. 87 of WO2001/047904, which was obtained from slurrying the evaporated reaction mixture in water and isopropanol.

### Example 6: Relative Bioavailability in Male Mice

The relative bioavailability of crystalline and different salt forms versus suspended IM-250 (derived from a DMSO stock solution) after single dose oral administration in male C57bl/6 mice was examined. Animals (3 per group) had food withdrawn approximately 2 h before administration. The suspension was prepared from a DMSO stock solution, which was diluted 1:20 with 0.5% HPMC in PBS, ultrasonificated and orally administered with a gavage volume of 5 mL/kg. The suspensions of crystalline and different salt forms were prepared directly by adding powder to 0.5% HPMC in PBS, ultrasonificated and orally administered with a gavage volume of 5 mL/kg. Blood samples were taken at 0.5 h, 1 h, 2 h, 5 h, 12 h and 24 h via capillary microsampling and bioanalytics was measured via non-chiral LC-MS. The area-under-curve (AUC_{0-24 h}) and relative bioavailability towards the IM-250 suspension was calculated. The doses for the salts were adjusted to 10 mg/kg free IM-250.

FIG. 12 shows the blood concentration over time. The following data (Table 6) was obtained:

TABLE 6
Area-under-curve (AUC_{0-24 h}) and relative
bioavailability for IM-250 in male mice

| | AUC_{0-24 h} | relative |
|---|---|---|
| Sample | (ng/mL*h) | bioavailability (%) |
| suspended IM-250 from DMSO | 66406 | — |
| (reference standard) | | |
| IM-250 Free base Form I | 7683 | 12 |
| IM-250 Napadisylate | 48241 | 73 |
| IM-250 HCl salt | 68289 | 103 |

### Example 7: Chemical and Physical Stability

The chemical and physical accelerated stability of the crystalline material was studied in order to anticipate potential stability issues upon storage or ageing. Crystals were stored for 1 month at 40° C./75% relative humidity (RH) and were also stored for 1 month at 60° C. (and % HR<10% RH). The chemical stability was assessed by HPLC by external standardization versus a freshly prepared (non-stressed) standard solution. For that purpose, six samples (exactly weighted) are stored for each condition: 3 for the 2-weeks stability and 3 for the 4-weeks stability. The physical stability was also assessed by XRPD, DSC analyses by comparing the XRPD and DSC profiles of stressed samples to those of a non-stressed sample. For that purpose, two more samples (one per time point) are stored in each condition (respectively 40° C./75% RH and 60° C.).

### Chemical Stability Results

The results (3 independent assays) of the HPLC analyses (UV at 285 nm) for IM-250 Free Base Form I are displayed in Table 7.

TABLE 7
Chemical degradation assessment after
temperature and humidity stress

| Stress | Time | % of unchanged | Impurity profile compared to |
|---|---|---|---|
| condition | point | compound left | standard profile |
| 40° C./ | 2 wk | 99.4 | Similar to standard profile |
| 75% RH | | (% CV = 0.3%) | No additional impurity |
| | 4 wk | 100.2 | Similar to standard profile |
| | | (% CV = 1.5%) | No additional impurity |
| 60° C. | 2 wk | 99.6 | Similar to standard profile |
| | | (% CV = 1.2%) | No additional impurity |
| | 4 wk | 101.4 | Similar to standard profile |
| | | (% CV = 0.6%) | No additional impurity |

% of unchanged compound left is determined versus standard solutions Container: open (only for 40° C./75% RH storage) glass vials protected from light

Given these results, the IM-250 Free Base Form I can be considered to be chemically stable as a bulk powder, at least 4 weeks after storage at 40° C./75% RH and at 60° C.

### Physical Stability Results

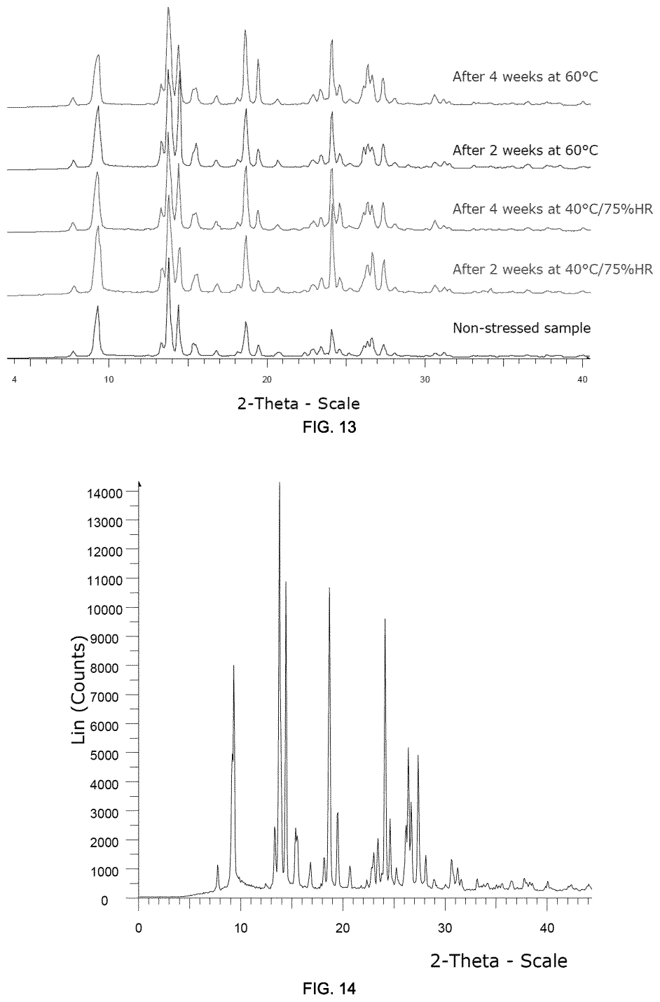

Table 8 displays the physical DSC characterization results of the bulk sample of IM-250 Free Base Form I stored for 2 and 4 weeks at 40° C./75% RH and at 60° C. compared to the initial characterization. The XRPD diffractograms under stress conditions were similar to the initial diffractogram. FIG. 13 displays staggered XRPD patterns from IM-250 Free Base Form I of a non-stressed sample and of samples stored for 2 and 4 weeks at 40° C./75% RH and at 60° C.

TABLE 8
Physical stability assessment of IM-250 Free Base
Form I by DSC after temperature and humidity stress

| | | | Temperature | | Enthalpy |
|---|---|---|---|---|---|
| Stress | Time | | (° C.) | | transition |
| condition | point | Transition | Onset | Peak | (J/g) |
| none | initial | Strong endotherm | 163 | 164 | −83 |
| | analysis | (melting) | | | |
| 40° C./ | 2 wk | Strong endotherm | 163 | 165 | −77 |
| 75% RH | | (melting) | | | |
| | 4 wk | Strong endotherm | 163 | 165 | −80 |
| | | (melting) | | | |
| 60° C. | 2 wk | Strong endotherm | 163 | 164 | −85 |
| | | (melting) | | | |
| | 4 wk | Strong endotherm | 163 | 165 | −80 |
| | | (melting) | | | |

In conclusion, the IM-250 Free Base Form I can be considered to be physically stable as a bulk powder, at least 4 weeks after storage at 40° C./75% RH and at 60° C.

### Example 8: ICH Stability Testing

The long term and accelerated chemical and physical stability was assessed at a Contract Manufacturing Organization. IM-250 HCl salt samples were primary packaged in double PE bags (50 μm, Semadeni, e.g. cat. #2439 with each tag tied with plastic twist). The secondary package was a HDPE-drum closed with a HDPE screw cap (CurTec). The storage conditions were 25±2° C./60±5% relative humidity and 40±2° C./75±5% relative humidity. The obtained results were as displayed in Table 9.

TABLE 9

| | | 6 months | 6 months |
|---|---|---|---|
| Test | Initial | 25° C. | 40° C. |
| Color and apperance | white to off- | white to off- | white to off- |
| | white solid | white solid | white solid |
| Purity (HPLC area %) | 99.2% | 99.2% | 99.1% |
| Assay (HPLC, % w/w, as-is) | 98.5% | 97.9% | 98.6% |
| Chiral purity (HPLC area %) | 99.8% | 99.9% | 99.9% |
| Water content (% w/w) | 0.08% | 0.08% | 0.10% |
| Identity by XRPD | conforms to | not tested | conforms to |
| | reference | | reference |

In conclusion, the IM-250 HCl salt can be considered to be physically stable as a bulk powder, at least 6 month after storage at 25° C. and at 40° C.

### Example 9: Synthesis of Deuterated IM-250 Free Base (d3-IM-250 Free Base)

### Step 1:1-Bromopropan-2-one-1,1,3,3,3-d₅ (9a)

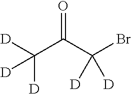

Br₂ (2.5 g, 15 mmol) was added to propan-2-one-d₆ (2.0 g, 31 mmol) at rt and after stirring for 2 h, the mixture was used in next step immediately.

### Step 2: N-Methyl-4-(methyl-d₃)thiazol-2-amine (9b)

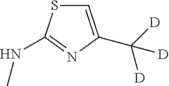

To a solution of compound 9a in EtOH (20 mL) was added 1-methylthiourea (1.4 g, 15 mmol) at 75° C. and after stirring for 2 h, a saturated NaHCO₃ solution was added. The mixture was extracted with EA (2×20 mL). The combined organic layer was dried over Na₂SO₄, filtered, concentrated and then purified by FCC (EA:PE=1:1) to give compound 9b.

### Step 3:5-Bromo-N-methyl-4-(methyl-d₃)thiazol-2-amine (9c)

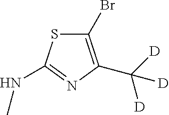

To a solution of compound 9b (400 mg, 3.0 mmol) in CHCl₃ (4 mL) was added Br₂ (740 mg, 4.7 mmol) at rt and after stirring overnight, water (10 mL) was added. The pH was adjusted to 8 with a saturated NaHCO₃ solution. The mixture was extracted with CHCl₃ (2×10 mL). The combined organic layer was washed with brine (10 mL), dried over Na₂SO₄, filtered and concentrated to give compound 9c as a solid.

### Step 4: N-Methyl-4-(methyl-d₃)-5-(methylthio)thiazol-2-amine (9d)

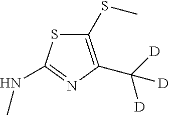

To a solution of compound 1c (350 mg, 1.6 mmol) in 1,4-dioxane (4 mL) was added MeSNa (230 mg, 3.2 mmol) at rt. After stirring overnight, the mixture was evaporated to get an oil, which was then purified by FCC (EA:PE=1:1) to give compound 9d as a yellow solid.

### Step 5:2-(2′,5′-Difluoro-[1,1′-biphenyl]-4-yl)-N-methyl-N-(4-(methyl-d₃)-5-(methylthio)thiazol-2-yl) acetamide (9e)

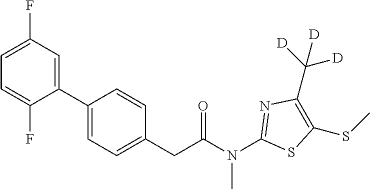 [SMILES: [2H]C([2H])([2H])c1nc(N(C)C(=O)Cc2ccc(-c3cc(F)ccc3F)cc2)sc1SC]

To a solution of 2-(2′,5′-difluoro-[1,1′-biphenyl]-4-yl)acetic acid (140 mg, 0.56 mmol), HATU (322 mg, 0.85 mmol) and Et₃N (171 mg, 0.85 mmol) in CH₂Cl₂ (2.0 mL) was added compound 9d (100 mg, 0.56 mmol) at rt. After stirring overnight, the mixture was washed with water (2×2.5 mL). The organic layer was dried over Na₂SO₄, filtered, concentrated and purified by FCC (PE:EA=2:1) to give compound 9e as a white solid.

### Step 6:2-(2′,5′-Difluoro-[1,1′-biphenyl]-4-yl)-N-methyl-N-(4-(methyl-d₃)-5-(methylsulfinyl)thiazol-2-yl)acetamide (9f)

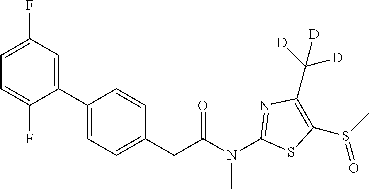 [SMILES: [2H]C([2H])([2H])c1nc(N(C)C(=O)Cc2ccc(-c3cc(F)ccc3F)cc2)sc1S(C)=O]

To a solution of compound 9e (180 mg, 0.44 mmol) in CH₂Cl₂ (1 mL) was added meta-chloroperoxybenzoic acid (76 mg, purity 85%). The mixture was stirred at rt for 20 min, partitioned between CH₂Cl₂ and 5 percent sodium carbonate solution. The organic phase was washed with brine, dried over Na₂SO₄, filtered, concentrated and purified by FCC (PE:EA=1:2) to give compound 9f as a white solid.

### Step 7: tert-Butyl ((2-(2-(2′,5′-difluoro-[1,1′-biphenyl]-4-yl)-N-methylacetamido)-4-(methyl-d₃)thiazol-5-yl)(methyl)(oxo)-I₆-sulfaneylidene)carbamate (9g)

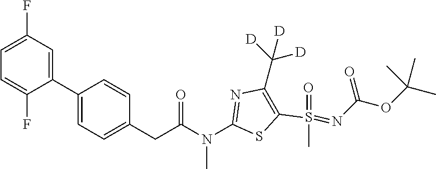 [SMILES: [2H]C([2H])([2H])c1nc(N(C)C(=O)Cc2ccc(-c3cc(F)ccc3F)cc2)sc1S(C)(=O)=NC(=O)OC(C)(C)C]

MgO (57 mg, 1.40 mmol), tert-butyl carbamate (83 mg, 0.70 mmol), Rh₂ (OAc) 4 (15 mg, 33 μmol) and (diacetoxy) iodobenzene (171 mg, 0.52 mmol) were added to a solution of compound 9f (150 mg, 0.35 mmol) in CH₂Cl₂ (2.5 mL). The mixture was stirred at 40° C. overnight, cooled to rt and filtered through a pad of celite. The solvent was removed under reduced pressure and the crude product was purified by FCC (PE:EA=1:1) to give compound 9g as a white solid.

### Step 8:2-(2′,5′-Difluoro-[1,1′-biphenyl]-4-yl)-N-methyl-N-(4-(methyl-d₃)-5-(S-methylsulfon-imidoyl)thiazol-2-yl)acetamide (9h)

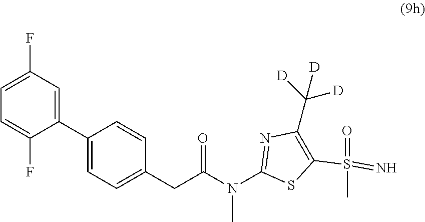 [SMILES: [2H]C([2H])([2H])c1nc(N(C)C(=O)Cc2ccc(-c3cc(F)ccc3F)cc2)sc1S(C)(=N)=O]

At ambient temperature, compound 9g (150 mg, 0.28 mmol) was added to a stirred solution of trifluoroacetic acid (2 mL) acid in CH₂Cl₂ (8 mL). Stirring was continued for 1 h, then the mixture was concentrated, resolved in CH₂Cl₂, washed with saturated NaHCO₃ (2×20 mL), dried over Na₂SO₄, filtered, concentrated and purified by prep-HPLC to give compound 9h as a white solid.

¹H-NMR (400 MHZ, DMSO-d₆) δ: 7.57 (d, J=7.2 Hz, 2H), 7.46-7.35 (m, 4H), 7.31-7.24 (m, 1H), 4.69 (s, 1H), 4.23 (s, 2H), 3.72 (s, 3H), 3.14 (s, 3H). MS: 439.1 [M+1]⁺.

### Step 9: (S)-2-(2′,5′-Difluoro-[1,1′-biphenyl]-4-yl)-N-methyl-N-(4-(methyl-d₃)-5-(S-methylsulfon-imidoyl)thiazol-2-yl)acetamide (Deuterated IM-250 Free Base, d3-IM-250 Free Base)

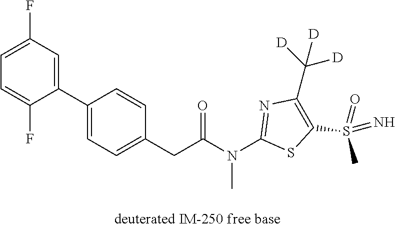 [SMILES: [2H]C([2H])([2H])c1nc(N(C)C(=O)Cc2ccc(-c3cc(F)ccc3F)cc2)sc1[S@@](C)(=N)=O]

The title compound was prepared by separation of the racemic mixture 9h by chiral SFC chromatography, using as stationary phase Chiralcel OJ and as mobile phase 55/45 vol. % CO₂/IPA and additional following data:
Instrument: SFC-150 (Thar, Waters)
Column: OJ 20×250 mm, 10 μm (Daicel)
Column temperature: 35° C.
Flow rate: 100 g/min
Back pressure: 100 bar
Detection wavelength: 214 nm
Cycle time: 3.7 min
Sample solution: 300 mg dissolved in 40 mL MeOH
Injection volume: 1.0 mL

The title compound deuterated IM-250 free base (d3-IM-250 free base) is obtainable with a purity of 99.7 area % by removing the mobile phase (solvent) of the first eluting enantiomer (retention time: 2.99 min) after evaporation of the CO₂ and removing the IPA by a rotary evaporator at 40° C.

XRPD analysis was conducted. FIG. 14 shows a XRPD pattern of deuterated IM-250 free base (d3-IM-250 free base). XRPD peaks were identified and are included in Table 10 below.

TABLE 10
XRPD Peak positions (°2Θ) and intensities

| °2-Theta (°2Θ) | Relative intensity (%) |
|---|---|
| 9.1 | 35 |
| 9.3 | 56 |
| 13.3 | 17 |
| 13.7 | 100 |
| 14.4 | 76 |
| 15.3 | 17 |
| 15.5 | 15 |
| 18.2 | 10 |
| 18.6 | 75 |
| 19.4 | 20 |
| 23.0 | 11 |
| 23.4 | 14 |
| 24.1 | 67 |
| 24.6 | 19 |
| 26.2 | 17 |
| 26.4 | 36 |
| 26.7 | 23 |
| 27.4 | 34 |
| 28.1 | 10 |

TGA and DSC analysis were performed. FIG. 15 shows the TGA thermogram of deuterated IM-250 free base (d3-IM-250 free base). The TGA analysis showed an onset/endset temperature of 243/305° C. which can be attributed to the thermal decomposition. The DSC analysis (FIG. 16) revealed a strong exotherm transition with onset at about 163° C. and peak at 165° C. (transition enthalphy 85 J/g).

### Example 10: Synthesis of Deuterated IM-250 HCl Salt (d3-IM-250 HCl Salt)

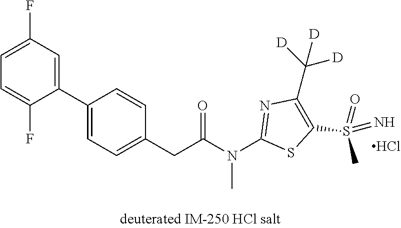 [SMILES: [2H]C([2H])([2H])c1nc(N(C)C(=O)Cc2ccc(-c3cc(F)ccc3F)cc2)sc1[S@@](C)(=N)=O ; Cl]

To a solution of deuterated IM-250 free base (850 mg) in acetone (50 mL) was added a quantity of 1N HCl corresponding to a 1:1 stoichiometry. The solution was homogenized at 40° C. before the solvents were removed in vacuo (50° C.), triggering the spontaneous crystallization of a white solid when only a few mL remained in the flask. In order to completely remove the water brought by the HCl addition, EtOH (2×5 mL) was added to the flask and the concentration at 50° C. was completed to dryness (only partial resolubilisation was observed during EtOH addition and stirring at 50° C.). More EtOH (5 mL) was then added to the flask, which was stirred at 50° C. and room temperature to resuspend the crystals. The supernatant was removed from the solid, which was then further dried under vacuum at 50-60° C. for about 3 h. White crystals of deuterated IM-250 HCl salt (d3-IM-250 HCl salt) were obtained with good yield.

XRPD analysis was conducted. FIG. 17 shows a XRPD pattern of IM-250 HCl salt (d3-IM-250 HCl salt). XRPD peaks were identified and are included in Table 11 below.

TABLE 11
XRPD Peak positions (°2Θ) and intensities

| °2-Theta (°2Θ) | Relative intensity (%) |
|---|---|
| 9.2 | 27 |
| 11.3 | 13 |
| 11.9 | 15 |
| 13.8 | 100 |
| 14.2 | 23 |
| 16.5 | 32 |
| 16.9 | 29 |
| 17.4 | 13 |
| 17.8 | 90 |
| 18.3 | 30 |
| 19.5 | 17 |
| 19.8 | 28 |
| 21.0 | 25 |
| 21.3 | 24 |
| 21.8 | 29 |
| 22.9 | 54 |
| 24.0 | 18 |
| 24.3 | 14 |
| 25.1 | 33 |
| 26.3 | 37 |
| 26.9 | 10 |
| 27.2 | 18 |
| 27.5 | 11 |
| 27.8 | 36 |
| 32.0 | 10 |
| 33.2 | 12 |

TGA and DSC analysis were performed. FIG. 18 shows an overlay of the DSC and TGA thermogram of deuterated IM-250 HCl salt (d3-IM-250 HCl salt). The TGA analysis (right curve) showed a mass loss of 7.8% upon heating with an onset/endset temperature of 149/167° C. before the main thermal decomposition can be detected with an onset temperature of 225° C. The 7.8% mass loss can be attributed to the departure of the HCl moiety. The DSC analysis revealed no 10 true melting point, rather a wide endotherm with onset at about 188° C. and peak at 194° C. (transition enthalphy −15 J/g).

### Example 11: Synthesis of Deuterated IM-250 Napadisylate Salt (d3-IM-250 Napadisylate)

The deuterated IM-250 Napadisylate salt form of compounds of the Formula (I) can be prepared similarly, by preparing the deuterated free base form as described above followed by conversion into the napadisylate salt as described above.

### Example 12: Relative Bioavailability and Brain Exposure of d3-IM-250 HCl Salt Versus IM-250 HCl Salt in Male Mice

The relative bioavailability of suspended crystalline deuterated IM-250 HCl salt (d3-IM-250 HCl salt) versus suspended IM-250 HCl salt after single dose oral administration in male C57bl/6 mice (ca. 8 weeks old) was examined. Animals (3 per group) had food withdrawn approximately 2 h before administration of 10 mg/kg of the test items. The suspensions were prepared directly by adding powder to 0.5% HPMC in PBS, ultrasonificated and orally administered with a gavage volume of 5 mL/kg. Blood samples (20 μL) were taken at 0.5 h, 1 h, 2 h, 5 h, 12 h and 24 h via capillary microsampling, collected from the tail vein into Li-heparin tubes. The samples were frozen on dry ice within 1-2 min of sampling and stored at −20° C. until LC-MS/MS analysis measured via non-chiral LC-MS. After 24 h post dose, animals were sacrificed and perfused with PBS until PBS was transparent and brain was collected and stored at −20° C. until processed for LC-MS analysis to determine the brain/blood exposure. The peak blood concentration (Cₘₐₓ), elimination half-life (t_{1/2}), area-under-curve (AUC_{0-24 h}) and blood/brain ratio (as an easily obtainable surrogate parameter for nerve tissue exposure) was determined. The following data (Table 12) was obtained:

TABLE 12
Effect of deuteration for the IM-250 HCl
salt in male mice on PK parameters

| Sample | IM-250 HCl salt | d3-IM-250 HCl salt |
|---|---|---|
| Cₘₐₓ [ng/mL] | 1210 | 2570 |
| t_{1/2} [h] | 5.7 | 6.9 |
| AUC₀₋₂₄ [ng/mL*h] | 14400 | 28600 |
| brain level @ 24 h [ng/g] | 175 | 568 |
| blood/brain ratio | 0.60 ± 0.20 | 0.49 ± 0.10 |

### CONCLUSION

Although the AUC₀₋₂₄ is somehow lower for IM-250 HCl salt compared to Example 6 in this experiment, selective deuteration at the 4-methyl position of the thiazole ring further improves the PK parameters, as evident within this matched-pair comparison for Cₘₐₓ, t_{1/2} and AUC₀₋₂₄. A beneficial and long lasting blood/brain ratio was obtained for both compounds.

1. A crystalline form of a compound according to Formula (I) 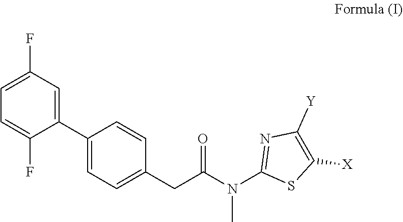
wherein X is selected from 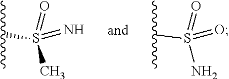
Y is selected from CH₃ and CD₃;
or a pharmaceutically acceptable salt, co-crystal, hydrate or solvate thereof.

2. A crystalline form of the compound according to Formula (I) of claim 1, wherein
X is 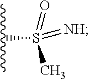 [SMILES: *[S@@](C)(=N)=O]
and
Y is selected from CH₃ and CD₃;
which is present in the form of a HCl salt or a hydrate or solvate thereof.

3. A crystalline form of a compound according to claim 1, selected from compounds with the following structure: 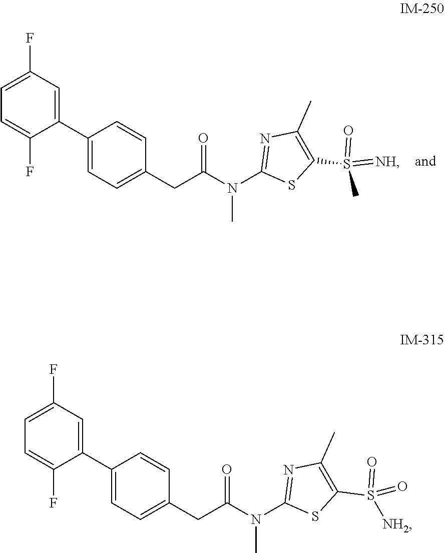 [SMILES: Cc1nc(N(C)C(=O)Cc2ccc(-c3cc(F)ccc3F)cc2)sc1[S@@](C)(=N)=O ; Cc1nc(N(C)C(=O)Cc2ccc(-c3cc(F)ccc3F)cc2)sc1S(N)(=O)=O]
wherein the crystalline form of compound IM-315 are characterized by having a melting point of 197° C. (±5° C.),
or a pharmaceutically acceptable salt, co-crystal, hydrate or solvate thereof.

4. A crystalline form of a compound IM-250 and IM-315 according to claim 3, selected from the following crystalline forms with an X-ray powder diffractogram comprising characteristic peaks (±0.2 degrees 2θ) at
IM-250 Free Base Form I: 9.2, 13.7 and 18.7 degrees,
IM-250 Free Base Form III: 9.7, 12.3 and 15.6 degrees,
IM-315:6.4, 12.5 and 18.3 degrees,
as determined on a diffractometer using Cu-Kα radiation at a wavelength of 1.54 Å.

5. A crystalline form of a compound IM-250 according to claim 1, which is present as a salt, selected from salt forms with the following structure:

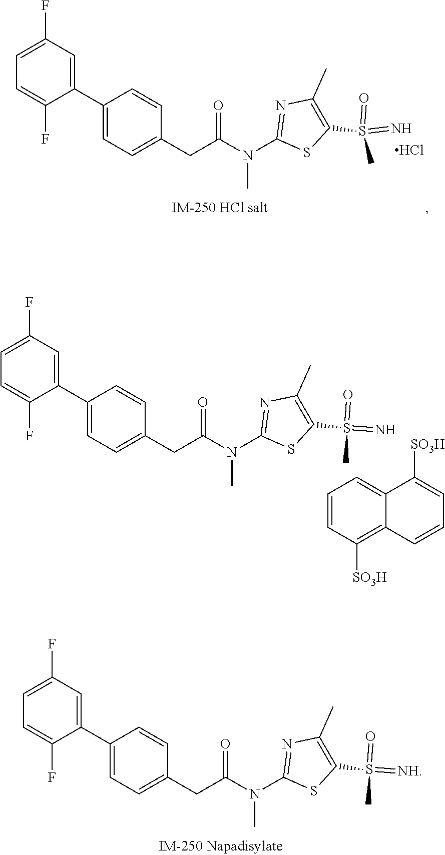

6. A crystalline form according to claim 2, which is present as a hydrochloride salt wherein hydrochloride and (S)-2-(2′,5′-difluoro-[1,1′-biphenyl]-4-yl)-N-methyl-N-(4-methyl-5-(S-methylsulfonimidoyl)thiazol-2-yl)acetamide are present in a 1:1 molar ratio.

7. A crystalline form according to claim 5, which are characterized by an X-ray powder diffractogram comprising at least 4 of the following peaks (±0.2 degrees 2θ):
IM-250 HCl salt: 13.7, 17.0, 17.7, 19.8, 21.8 and 22.8,
IM-250 Napadisylate salt: 9.1, 14.5, 15.6, 18.1, 19.1 and 20.9,
as determined on a diffractometer using Cu-Kα radiation at a wavelength of 1.54 Å.

8. A crystalline form of a compound according to claim 1, with the following structure: 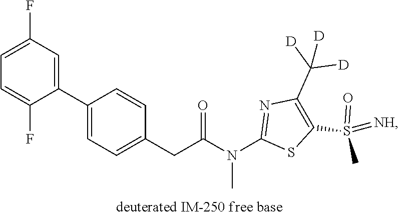 [SMILES: [2H]C([2H])([2H])c1nc(N(C)C(=O)Cc2ccc(-c3cc(F)ccc3F)cc2)sc1[S@@](C)(=N)=O]
with an X-ray powder diffractogram comprising characteristic peaks (±0.2 degrees 2θ) at 9.3, 13.7 and 18.6 degrees, as determined on a diffractometer using Cu-Kα radiation at a wavelength of 1.54 Å.

9. A crystalline form of a compound according to claim 1, with the following structure: 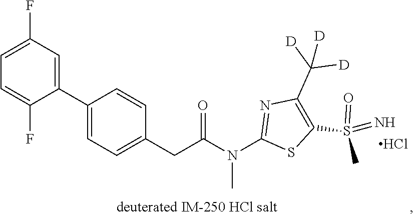 [SMILES: [2H]C([2H])([2H])c1nc(N(C)C(=O)Cc2ccc(-c3cc(F)ccc3F)cc2)sc1[S@@](C)(=N)=O ; Cl]
with an X-ray powder diffractogram comprising characteristic peaks (±0.2 degrees 2θ) at 13.8, 17.8 and 21.8 degrees, as determined on a diffractometer using Cu-Kα radiation at a wavelength of 1.54 Å.

10. A crystalline form according to claim 3, having an XRPD pattern substantially as shown in FIG. 1, 3, 5, 7, 8, 10, 14 or 17.

11. A pharmaceutical composition comprising a therapeutically effective amount of a crystalline form of Formula (I) according to claim 1, or a pharmaceutically acceptable salt, co-crystal, hydrate or solvate thereof, and a pharmaceutically acceptable excipient.

12. A method for prophylaxis or treatment of a herpes simplex infection or -mediated disorder, comprising administering the pharmaceutical composition according to claim 11 to a patient in need thereof.

13. A process for preparing a solid compound of the following structure: 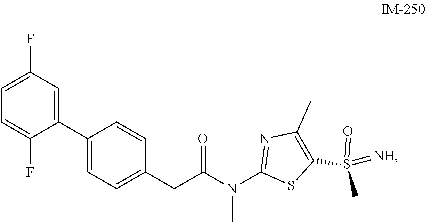 [SMILES: Cc1nc(N(C)C(=O)Cc2ccc(-c3cc(F)ccc3F)cc2)sc1[S@@](C)(=N)=O]
or a pharmaceutically acceptable salt, co-crystal, hydrate or solvate thereof, comprising
(a) contacting a compound P2b: 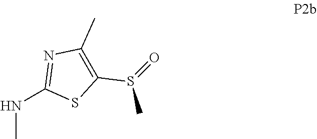
with a compound: 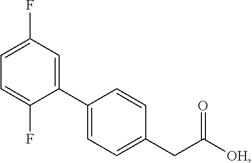 [SMILES: O=C(O)Cc1ccc(-c2cc(F)ccc2F)cc1]
(b) contacting a compound P2c: 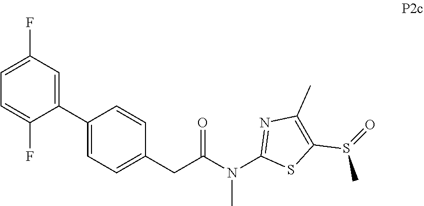 [SMILES: Cc1nc(N(C)C(=O)Cc2ccc(-c3cc(F)ccc3F)cc2)sc1[S@](C)=O]
with Rh₂(OAc)₄, tert-butyl carbamate, magnesium oxide and (diacetoxy) iodobenzene,
under conditions sufficient to form a compound P2d: 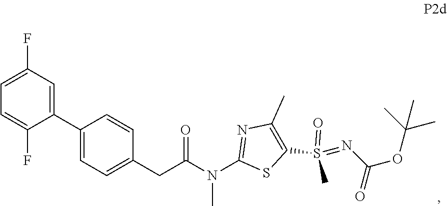 [SMILES: Cc1nc(N(C)C(=O)Cc2ccc(-c3cc(F)ccc3F)cc2)sc1[S@](C)(=O)=NC(=O)OC(C)(C)C]
(c) deprotecting compound P2d under conditions sufficient to form a compound with the structure 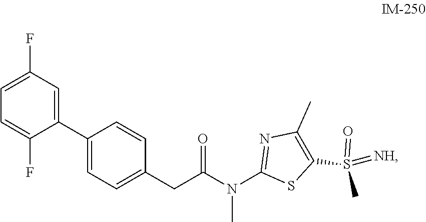 [SMILES: Cc1nc(N(C)C(=O)Cc2ccc(-c3cc(F)ccc3F)cc2)sc1[S@@](C)(=N)=O]
and
(d) optionally converting the compound IM-250 into a pharmaceutically acceptable salt, co-crystal, hydrate or solvate thereof.

14. The process of claim 13, wherein the compound P2d: 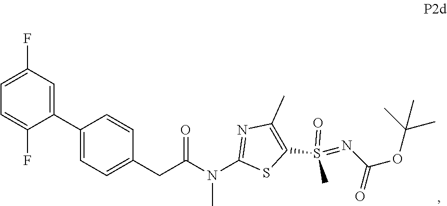 [SMILES: Cc1nc(N(C)C(=O)Cc2ccc(-c3cc(F)ccc3F)cc2)sc1[S@](C)(=O)=NC(=O)OC(C)(C)C]
is deprotected with hydrochloric acid to form a HCl salt according to compound IM-250 HCl salt: 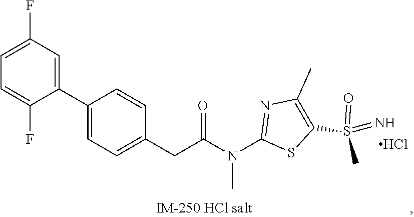 [SMILES: Cc1nc(N(C)C(=O)Cc2ccc(-c3cc(F)ccc3F)cc2)sc1[S@@](C)(=N)=O ; Cl]
or
is deprotected with 1,5-naphthalenedisulfonic acid tetrahydrate to form a napadisylate salt according to compound IM-250 Napadisylate salt:

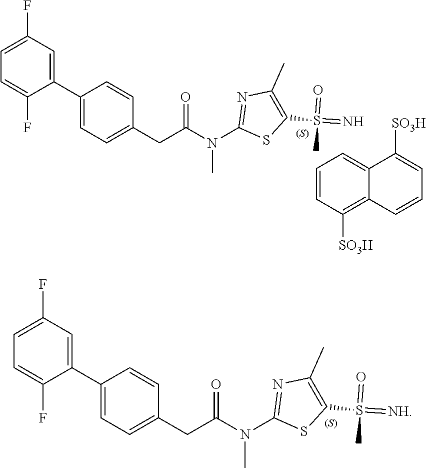

15. The process according to claim 13, further including a step of deuteration to obtain the deuterated analogs of the solid compounds and salts, wherein the deuterated analogs have to following structures: 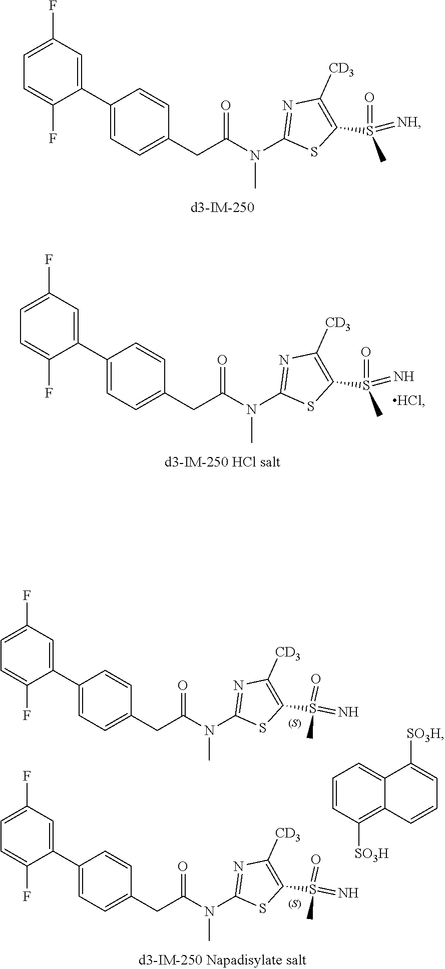
or co-crystal, hydrate or solvate thereof.
